(19) United States
(12) Patent Application Publication
(10) Pub. No.: US 2003/0068275 A1
(43) Pub. Date: Apr. 10, 2003

(54) Coordination complexes for detecting analytes, and methods of making and using the same

(76) Inventors: Stephen J. Lippard, Cambridge, MA (US); Scott Hilderbrand, Cambridge, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/228,821

(22) Filed: Aug. 27, 2002

Related U.S. Application Data
(60) Provisional application No. 60/315,232, filed on Aug. 27, 2001.

(51) Int. Cl. A61K 49/00; C07F 5/00

(52) U.S. Cl. 424/9.36; 424/9.6; 534/15; 534/16

(57) ABSTRACT

The present invention is directed, in part, to coordination complexes for detecting analytes, and methods of making and using the same.

### CROSS-REFERENCE TO RELATED APPLICATIONS

This application claims the benefit of U.S. Provisional Application No. 60/315,232, filed Aug. 27, 2001, the contents of which are hereby incorporated by this reference in their entirety.

### INTRODUCTION

I. Fluorescent Sensors

Fluorescence technology has revolutionized cell biology and many areas of biochemistry. In certain instances, fluorescent molecules may be used to trace molecular and physiological events in living cells. Certain sensitive and quantitative fluorescence detection devices have made fluorescence measurements an ideal readout for in vitro biochemical assays. In addition some fluorescence measurement systems may be useful for determining the presence of analytes in environmental samples. Finally, because certain fluorescence detection systems are rapid and reproducible, fluorescence measurements are often critical for many high-throughput screening applications.

The feasibility of using fluorescence technology for a particular application is often limited by the availability of an appropriate fluorescent sensor. There are a number of features that are desirable in fluorescent sensors, some of which may or may not be present in any particular sensor. First, fluorescent sensors should produce a perceptible change in fluorescence upon binding a desired analyte. Second, fluorescent sensors should selectively bind a particular analyte. Third, to allow concentration changes to be monitored, fluorescent sensors should have a K_{d} near the median concentration of the species under investigation. Fourth, fluorescent sensors, especially when used intracellularly, should produce a signal with a high quantum yield. Fifth, the wavelengths of both the light used to excite the fluorescent molecule (excitation wavelengths) and of the emitted light (emission wavelengths) are often important. If possible, for intracellular use, a fluorescent sensor should have excitation wavelengths exceeding 340 nm to permit use with glass microscope objectives and prevent UV-induced cell damage, and possess emission wavelengths approaching 500 nm to avoid altofluorescence from native substances in the cell and allow use with typical fluorescence microscopy optical filter sets. Sixth, ideal sensors should allow for passive and irreversible loading into cells. Finally, ideal sensors should exhibit increased fluorescence with increasing levels of analyte.

II. Nitric Oxide in Biological Systems

Since the discovery in the 1980s that nitric oxide (NO) is the endothelium-derived relaxing factor (EDRF), postulated biological roles for NO have continued to proliferate. For example, in addition to cardiovascular signaling, NO also seems to function as a neurotransmitter that may be important in memory and as a weapon to fight infection when released by immune system macrophages. Uncovering these roles and deciphering their implications is complicated by the array of reactions that this gaseous molecule undergoes. In a biological environment, NO can react with a host of targets, including dioxygen, oxygen radicals, thiols, amines and transition metal ions. Some of the products formed, such as NO₂ and NO⁺, are pathophysiological agents, whereas others, such as S-nitrosothiols, may in fact themselves be NO-transfer agents. Transition metal centers, especially iron in oxyhemoglobin, can rapidly scavenge free NO, thereby altering the amount available for signaling purposes.

The concentration-dependent lifetime of NO as well as its ability to diffuse freely through cellular membranes further complicate the delineation of these various processes. With a lifetime of up to 10 min under some conditions and a diffusion range of 100-200 μm, the biological action of NO can be distant from its point of origin. A diffusional spread of 200 μm corresponds to a volume containing approximately 2 million synapses.

A variety of analytical methods are available to monitor aspects of NO in biology, each having certain limitations. The Griess assay, for instance, is useful for estimating total NO production, but it is not very sensitive, cannot give real-time information and only measures the stable oxidation product nitrite. Although more sensitive and selective for NO, the chemiluminescent gas phase reaction of NO with ozone requires purging aqueous samples with an inert gas to strip NO into an analyzer. It too is therefore incapable of monitoring intracellular NO. Electrochemical sensing using microsensors provides in situ real-time detection of NO; the only spatial information obtained, however, is directly at the electrode tip and is therefore influenced by the placement of the probe.

Fluorescent NO sensors include DAF (diaminofluorescein) and DAN (2,3-diaminonaphthalene), the aromatic vicinal diamines of which react with nitrosating agents (NO⁺ or NO₂) to afford fluorescent triazole compounds. DAF compounds can report intracellular NO, but their sensing ability relies on NO autoxidation products and not direct detection. A rhodamine-type fluorescent NO indicator similarly senses autoxidation products.

Fluorescent nitric oxide cheletropic traps (FNOCTs) are fluorescent versions of molecules that have been used as EPR spin probes and do react directly with NO. The initially formed nitroxide radical species formed are not fluorescent, however. Addition of a common biological reductant such as ascorbic acid is required to reduce the nitroxide and display increased fluorescence intensity.

The present invention is directed in part to fluorescent sensors for low molecular weight, biologically-relevant molecules, e.g., nitric oxide, based upon various fluorophores having a metal binding domain. In part, the present invention is directed to fluorescent sensors, and methods of making and using the same, that allow for the detection of nitric oxide and, optionally, quantification of its concentration.

### SUMMARY OF THE INVENTION

In one aspect, the present invention is directed to coordination complexes that change their fluorescence properties in the presence of certain chemical moieties, and methods of making and using the same. In part, the present invention is directed to coordination complexes, and methods of making and using the same, containing fluorophores that, upon exposure to an analyte, exhibit different fluorescence properties than when the analyte is not present, optionally an increase in quantum yield or fluorescence intensity of such fluorophores (as opposed to a decrease). Such change in certain embodiments may be attributable to coordination of the analyte to a metal ion in the coordination complex.

In one aspect, the present invention is directed towards coordination complexes comprising a number of Lewis base moieties that are coordinated to a metal ion and form a generalized equatorial plane and at least one ligand that is axial to that plane, wherein the axial ligand comprises a fluorophore with a metal binding domain, with the complex being capable of exhibiting a change in fluorescence upon exposure to an analyte. In certain embodiments, the fluorescence increases upon such exposure.

In certain embodiments, the present invention is directed towards a coordination complex composed of a metal ion, a macrocycle, and a fluorophore with a metal binding domain, with the complex being capable of exhibiting a change in fluorescence upon exposure to an analyte. In certain embodiments, the fluorescence increases upon such exposure.

In another embodiment, the present invention teaches that a coordination complex comprising two metals, a number of bidentate ligands, and a fluorophore with a metal binding domain, with the complex being capable of exhibiting a change in fluorescence upon exposure to an analyte. In certain embodiments, the fluorescence increases upon such exposure.

In certain embodiments, the analyte is NO.

The subject compositions, and methods of making and using the same, may achieve a number of desirable results and features, one or more of which (if any) may be present in any particular embodiment of the present invention: (i) the subject coordination complexes bind, optionally reversibly, a desired analyte with a concomitant change in the fluorescence properties; (ii) a general schematic whereby a variety of useful coordination complexes, varying optionally in the metal ion, ligands, or fluorophore, may be constructed for use as sensors for certain analytes; (iii) the subject coordination complexes selectively bind certain analytes, optionally reversibly; (iv) coordination complexes exhibit an increase in quantum yield (as opposed to a decrease) upon coordination of an analyte of interest; (v) coordination complexes may be capable of in vivo and other diagnostic use; and (vi) novel chelating ligands containing fluorophores.

In certain embodiments, the subject invention is directed to coordination complexes generally represented by the moiety of Formula 1: {M(MC)(V—F)}, wherein: MC represents a macrocycle that is capable of coordinating a metal ion through at least two Lewis basic atoms; M is a metal ion; V is a metal binding domain that is capable of forming a coordinate bond with M; and F is a fluorophore. In certain embodiments, a coordination complex of Formula 1 may be charged. In certain embodiments, a coordination complex of Formula 1 may have additional components, such as other ligands, counter-ions, molecules of solvation and the like. In certain embodiments, V—F may be tethered to the macrocycle MC through a covalent tether. In certain embodiments, the macrocycle MC may be derivatized to enhance analyte binding, the reversibility of analyte binding, and other properties of the resulting coordination complex.

In certain embodiments, the subject invention is directed to coordination complexes generally represented by the moiety of Formula 7: {Mₘ(W)ₙ(V—F)ₚ}, wherein independently for each occurrence: W represents a ligand which is capable of coordinating one or more metal ions through at least two Lewis basic atoms; M is a metal ion; V is a metal binding domain that is capable of forming a coordinate bond with M; F is a fluorophore; m is at least 2, and n and p are each independently 1,2,3 or 4. In certain embodiments, a coordination complex of Formula 7 may be charged. In certain embodiments, the coordination complex of Formula 7 may have additional components, such as other ligands, counter-ions, molecules of solvation and the like. In certain embodiments, V—F may be tethered to W or another ligand of the coordination complex through a covalent tether. In certain embodiments, W and other ligands of the coordination complex may be derivatized to enhance analyte binding, the reversibility of analyte binding, and other properties of the resulting coordination complex.

The above and further features and advantages of the invention are described in the following specification. The claims appended hereto are hereby incorporated into this Summary in their entirety.

In certain embodiments, the subject coordination complexes of the present invention have the structures described in greater detail below, all of which structures are hereby incorporated by reference in their entirety into this Summary to describe the present invention. In addition, the claims appended hereto are hereby incorporated into this Summary in their entirety.

In another aspect, the present invention provides methods of making the subject coordination complexes and ligands.

In another aspect, the present invention provides novel fluorophores with a metal binding domain (e.g., V—F from Formula 1 or Formula 7), and methods of making and using the same. In still other embodiments, the present invention provides novel ligands in which a fluorophore with a metal binding domain is tethered to a macrocycle, bidentate ligand or other ligands.

In another aspect, the subject invention involves methods of using the subject coordination complexes to detect, and optionally to quantify concentrations of, certain analytes in a sample. The detection methods rely on the change observed in the fluorescence of the subject coordination complexes upon exposure to an analyte of interest. In certain embodiments, any such change observed may be attributable to binding of one or more analyte molecules to one or more metal ions of a subject coordination complex and dissociation of the metal binding domain of the ligand V—F, as discussed in greater detail below. Any change observed, both positive and negative, and including, for example, a change in the emission wavelength, the excitation wavelength, and the quantum yield, may be used to detect analyte presence. The methods may be used in vivo to detect changes in intracellular concentrations of analytes with the appropriate coordination complexes. In addition, the present inventive methods provide for positive and negative controls. In certain embodiment, the methods may be used for continuous analysis of a sample.

In another aspect, the present invention is directed to methods of using the subject coordination complexes for diagnostic purposes. In certain instances, the subject compositions and methods may be used to detect, and optionally to quantify the concentration of, an analyte present in a patient. In certain embodiment, the methods may be used for continuous analysis of a patient. In certain embodiments, that analyte may be indicative of a disease or condition, or on treatment regimen for a disease or condition as opposed to a second treatment regimen, etc.

In another aspect, the present invention is directed to methods of using the subject coordination complexes for determining the presence of analytes in samples, including samples of environmental interest. In certain aspects, such samples may have a pH of approximately 3, 4 5, 6, 7, 8, 9, 10, 11, 12, or higher.

In other embodiments, this invention contemplates a kit including subject compositions, and optionally instructions for their use. Uses for such kits include, for example, diagnostic applications.

These embodiments of the present invention, other embodiments, and their features and characteristics, will be apparent from the description, drawings and claims that follow.

### BRIEF DESCRIPTION OF THE FIGURES

FIG. 1 depicts a synthetic strategy for preparing one ligand V—F, a rhodafluor containing a piperidine-like moiety, Rhodapip.

FIG. 2A. depicts the structures of cobalt(II)tetraphenylporphyrin (“Co(TPP)”) and the “Rhodapip” ligand prepared as shown in FIG. 1. The equation depicts the coordination chemistry that is believed to be the basis for the ability of the sensor to detect NO. B. depicts a mixture of Co(TPP) and Rhodapip in the absence (left tube) and presence (right tube) of excess NO.

FIG. 3. depicts a synthetic strategy for preparing [Co₂(μ-O₂CAr^{Tol})₄(dansylpiperazine)₂], an example of a coordination complex of Formula 7.

FIG. 4.A. The equation depicts the coordination chemistry that is believed to be the basis for the observed change in fluorescence upon exposure to excess NO. B. depicts [Co₂(O₂CAr^{Tol})₄(dansylpiperazine)₂] in the absence (left tube) and presence (right tube) of excess NO.

FIG. 5. depicts the fluorescence response as measured by excitation spectra of [Co₂(O₂CAr^{Tol})₄(dansylpiperazine)₂] after exposure to excess NO. The experimental conditions are described in the Exemplification section below.

### DETAILED DESCRIPTION OF THE INVENTION

In part, the present invention is directed to sensors that are coordination complexes and may be used to detect certain analytes using fluorescence. In certain embodiments, there is a positive change in fluorescence upon exposure of an analyte of interest to a subject composition. In certain embodiments, described in terms of a molecular switch, the subject metal complexes are in the “off” position in the absence of a specified analyte. Subsequent exposure to such an analyte turns the fluorescence “on”.

Without limiting the invention to a particular mechanism of action or otherwise circumscribing the scope of the teachings herein, it is believed that when coordinated to the metal ion quenching of the fluorescence of F in the ligand V—F is believed to be due primarily to photoinduced electron transfer (PET) and electronic energy transfer (EET). It is believed that upon exposure to an analyte of interest, such as NO, the ligand V—F is displaced from the metal ion, whereupon F is no longer in close proximity to the metal ion and is therefore no longer quenched. Thereupon, the ligand V—F fluoresces. Accordingly, to prepare coordination complexes that will serve as sensors, it will be necessary to take such quenching into account. For example, in those embodiments when the ligand V—F is tethered to the macrocycle of a subject coordination complex, the structure and geometry of the tether may need to be varied to give the coordination complex with the greatest change in fluorescence upon exposure to an analyte of interest.

I. Definitions

For convenience, before further description of the present invention, certain terms employed in the specification, examples and appended claims are collected here. These definitions should be read in light of the remainder of the disclosure and understood as by a person of ordinary skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used herein to mean “including but not limited to”. “Including” and “including but not limited to” are used interchangeably.

The term “macrocycle” or “MC” is art-recognized and refers to a molecule, often an organic one, that contains a ring moiety, usually having more than 12 atoms, capable of coordinating a metal ion. Some examples of macrocycles are porphyrins, pthalocyanines, corroles, sapphyrins, salens, acens, crown ethers, azacrown ethers, cyclams, and the like. Other examples of macrocycles are described in more detail below. In certain embodiments, a single ring moiety of the macrocycle contains all the Lewis base atoms capable of forming a coordinate bond with a single metal ion, such as is the case for a simple porphyrin or TACN. In certain other embodiments, there is an additional Lewis base moiety that is capable of forming a coordinate bond with the single metal ion and such additional Lewis base moiety is covalently attached to the ring.

The term “fluorophore” is art-recognized and refers to a molecule or moiety, generally a polyaromatic hydrocarbon or heterocycle, that has the ability to fluoresce. The ability to fluoresce, or “fluorescence”, of a fluorophore is generally understood to result from a three-stage process: (i) excitation, in which a photon is absorbed by the fluorophore, creating an excited electronic state in which the fluorophore has greater energy relative to the normal electronic state of the fluorophore; (ii) excited state lifetime, during which the fluorophore remains in the excited electronic state but also during which the energy of the state is partially dissipated; and (iii) emission, in which a photon of lower energy is emitted. Thus, a fluorophore absorbs a different wavelength of light (the “excitation wavelength”) than it emits (the “emission wavelength”). Examples of fluorophores are described in more detail below. The terms “excitation wavelength” and “emission wavelength” are well-known in the art. The term “quantum yield” is art-recognized and refers to the efficiency of photon emission by the fluorophore and is described in more detail below.

The terms “tether” or “covalent tether” are art-recognized and refer to a chemical moiety that covalently links two chemical moieties or molecules together. Such a tether may be flexible, so that the two molecules may move relative to one another, or have a constrained conformation, so that the two molecules are held in a fixed position relative to each other. The length of a tether may be varied in such a way as to confer a desired spacing between the two molecules. Examples of suitable tethers for use in the present invention are described in more detail below.

The terms “Lewis base” and “Lewis basic” are art-recognized and generally include a chemical moiety, a structural fragment or substituent, or single atom capable of donating a pair of electrons under certain conditions. It may be possible to characterize a Lewis base as donating a single electron in certain complexes, depending on the identity of the Lewis base and the metal ion, but for most purposes, however, a Lewis base is best understood as a two electron donor. Examples of Lewis basic moieties include uncharged compounds such as alcohols, thiols, and amines, and charged moieties such as alkoxides, thiolates, carbanions, and a variety of other organic anions. A Lewis base, when coordinated to a metal ion, is often referred to as a ligand. Further description of ligands relevant to the present invention is presented below.

The term “ligand” refers to a species that interacts in some fashion with another species. In one example, a ligand may be a Lewis base that is capable of forming a coordinate bond with a Lewis acid. In other examples, a ligand is a species, often organic, that forms a coordinate bond with a metal ion. Ligands, when coordinated to a metal ion, may have a variety of binding modes know to those of skill in the art, which include, for example, terminal (i.e., bound to a single metal ion) and bridging (i.e., one atom of the Lewis base bound to more than one metal ion).

The terms “Lewis acid” and “Lewis acidic” are art-recognized and refer to chemical moieties which can accept a pair of electrons from a Lewis base as defined above. Using the nomenclature of Lewis base and Lewis acid, it is understood in the art that a metal ion is most often a Lewis acid.

The term “chelating agent” refers to a molecule, often an organic one, and often a Lewis base, having two or more unshared electron pairs available for donation to a metal ion via at least two different atoms. The metal ion is usually coordinated by two or more electron pairs to the chelating agent. The terms, “bidentate chelating agent”, “tridentate chelating agent”, and “tetradentate chelating agent” refer to chelating agents having, respectively, two, three, and four electron pairs on two, three and four different atoms, respectively, capable of simultaneous donation to a metal ion coordinated by the chelating agent. Usually, the electron pairs of a chelating agent form coordinate bonds with a single metal ion; however, in certain examples, a chelating agent may form coordinate bonds with more than one metal ion, with a variety of binding modes being possible.

The term “coordination” refers to an interaction in which one multi-electron pair donor coordinatively bonds (is “coordinated”) to one metal ion.

The terms “coordinate bond” or “coordination bond” refer to an interaction between an electron pair donor and a coordination site on a metal ion leading to an attractive force between the electron pair donor and the metal ion. The use of these terms is not intended to be limiting, in so much as certain coordinate bonds may also be classified as having more or less covalent character (if not entirely covalent character) depending on the nature of the metal ion and the electron pair donor.

The term “metal binding domain” refers to a portion or all of a molecule that contains at least one Lewis base capable of forming a coordinate bond with a metal ion. For example and without limitation, a metal binding domain may consist of a function group such a carboxylate consisting of more than one atom, a bidentate ligand such as trien consisting of many atoms, or a single atom such as an oxide.

The term “coordination site” refers to a point on a metal ion that can accept an electron pair donated, for example, by a liquid or chelating agent.

The term “free coordination site” refers to a coordination site on a metal ion that is vacant or occupied by a species that is weakly donating. Such species is readily displaced by another species, such as a Lewis base.

The term “coordination number” refers to the number of coordination sites on a metal ion that are available for accepting an electron pair.

The term “coordination geometry” refers to the manner in which coordination sites and free coordination sites are spatially arranged around a metal ion. Some examples of coordination geometry include octahedral, square planar, trigonal, trigonal biplanar and others known to those of skill in the art. In certain coordination geometries, a coordination site may be identified as “axial” or “equatorial”. For example, for an general octahedral coordination geometry, there are four equatorial coordination sites and two axial coordination sites. In contrast, for a general square planar coordination geometry, there are four equatorial coordination sites and a single axial coordination site. In contrast, for a general trigonal biplanar coordination geometry, there are three equatorial coordination sites and two axial coordination sites. As one example, for a porphyrin macrocycle and an octahedral coordination geometry for the metal ion coordinated thereby, the four nitrogen atoms of the macrocycle are in the equatorial coordination sites, leaving two axial sites, one above and one below the plane of the macrocycle. As for all these coordination geometries, the actual structure of any subject coordination complex will deviate from the idealized coordination geometries described here, but it is often the case that the coordination geometry for the metal ion(s) in the complex may often be best described as belonging to one coordination geometry and not the others.

The term “complex” means a compound formed by the union of one or more electron-rich and electron-poor molecules or atoms capable of independent existence with one or more electronically poor molecules or atoms, each of which is also capable of independent existence. A “coordination complex” is one type of a complex, in which there is a coordinate bond between a metal ion and an electron pair donor. A metal ion complex is a coordination complex in which the metal ion is a metal ion. In general, the terms “compound,” “composition,” “agent” and the like discussed herein include complexes, coordination complexes and metal ion complexes. One example of a coordination complex is a macrocycle and a metal ion. As a general matter, the teachings of Advanced Inorganic Chemistry by Cotton and Wilkinson are referenced as supplementing the definitions herein in regard to coordination complexes and related matters.

In certain circumstances, a coordination complex may be understood to be composed of its constitutive components. For example, a coordination complex may have the following components: (i) one or more metal ions, which may or may not be the same atom, have the same charge, coordination number or coordination geometry and the like; and (ii) one or more Lewis bases that form coordinate bonds with the metal ion(s), such as a macrocycle. Examples of such Lewis bases include chelating agents and ligands.

If a coordination complex is charged, in that the metal ion and any Lewis bases, in the aggregate, are not neutral, then such a complex will usually have one or more counterions to form a neutral compound. Such counterions may or may not be considered part of the coordination complex depending on how the term coordination complex is used. Counterions generally do not form coordinate bonds to the metal ion, although they may be associated, often in the solid state, with the metal ion or Lewis bases that make up the coordination complex. Some examples of counterions include monoanions such as nitrate, chloride, tetraflurorborate, hexafluorophosphate, and monocarboxylates, and dianions such as sulfate. In some cases, coordination complexes themselves may serve as counterions to another coordination complex.

The same chemical moiety may be either a ligand or a counterion to a coordination complex. For example, the anionic ligand chloride may be either coordinately bound to a metal ion or may act as a counterion without any need for bond formation. The exact form observed for chloride in any coordination complex will depend on a variety of factors, including theoretical considerations, such as kinetic versus thermodynamic effects, and the actual synthetic procedures utilized to make the coordination complex, such as the extent of reaction, acidity, concentration of chloride. These considerations are applicable to other counterions as well.

Additionally, a coordination complex may be solvated. Solvation refers to molecules, usually of solvent and often water, that associate with the coordination complex in the solid state. Again, as for counterions, such solvation molecules may or may not be considered part of the coordination complex depending on how the term coordination complex is used.

The term “synthetic” refers to production by in vitro chemical or enzymatic synthesis.

The term “meso compound” is recognized in the art and means a chemical compound which has at least two chiral centers but is achiral due to a plane or point of symmetry.

The term “chiral” refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” refers to molecules with identical chemical constitution and containing more than one stereocenter, but which differ in configuration at only one of these stereocenters.

“Small molecule” is an art-recognized term. In certain embodiments, this term refers to a molecule which has a molecular weight of less than about 2000 amu, or less than about 1000 amu, and even less than about 500 amu.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

Moreover, the term “alkyl” (or “lower alkyl”) includes “substituted alkyls”, which refers to alkyl moieties having substituents replacing a hydrogen on one or more carbons of the hydrocarbon backbone. Such substituents may include, for example, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Exemplary substituted alkyls are described below. Cycloalkyls may be further substituted with alkyls, alkenyls, alkoxys, alkylthios, aminoalkyls, carbonyl-substituted alkyls, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas:

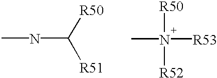

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

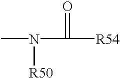

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

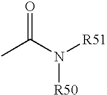

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

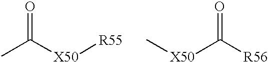

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and includes a moiety that may be represented by the general formula:

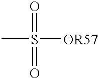

in which R57 is an electron pair, hydrogen, alkyl, cycloalkyl, or aryl.

The term “sulfate” is art recognized and includes a moiety that may be represented by the general formula:

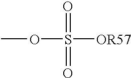

in which R57 is as defined above.

The term “sulfonamido” is art recognized and includes a moiety that may be represented by the general formula:

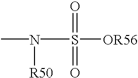

in which R50 and R56 are as defined above.

The term “sulfamoyl” is art-recognized and includes a moiety that may be represented by the general formula:

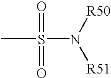

in which R50 and R5 1 are as defined above.

The term “sulfonyl” is art recognized and includes a moiety that may be represented by the general formula:

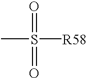

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

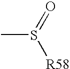

in which R58 is defined above.

The term “phosphoryl” is art recognized and includes moieties represented by the general formula:

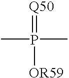

wherein Q50 represents S or O, and R59 represents hydrogen, a lower alkyl or an aryl. When used to substitute, e.g., an alkyl, the phosphoryl group of the phosphorylalkyl may be represented by the general formulas:

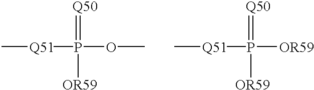

wherein Q50 and R59, each independently, are defined above, and Q51 represents O, S or N. When Q50 is S, the phosphoryl moiety is a “phosphorothioate”.

The term “phosphoramidite” is art recognized and includes moieties represented by the general formulas:

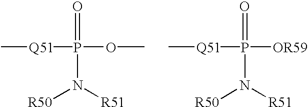

wherein Q51, R50, R51 and R59 are as defined above.

The term “phosphoramidite” is art recognized and includes moieties represented by the general formulas:

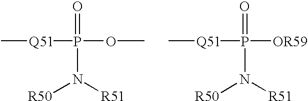

wherein Q51, R50, R51 and R59 are as defined above.

The term “phosphonamidite” is art recognized and includes moieties represented by the general formulas:

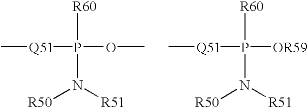

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compositions of the present invention may exist in particular geometric or stereoisomeric forms. In addition, certain compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive finctional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like. By the terms “amino acid residue” and “peptide residue” is meant an amino acid or peptide molecule without the —OH of its carboxyl group. In general the abbreviations used herein for designating the amino acids and the protective groups are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by Kopple, Peptides and Amino Acids 2, 33 (W. A. Benjamin Inc., New York and Amsterdam, 1966); examples of such side chains of the common amino acids are —CH₂CH₂SCH₃ (the side chain of methionine), —CH₂CH(CH₃)₂ (the side chain of leucine) or —H (the side chain of glycine).

The term “amino acid” is intended to embrace all compounds, whether natural or synthetic, which include both an amino functionality and an acid functionality, including amino acid analogs and derivatives. In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups. For instance, the subject compounds may include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers may be obtained in substantially pure form by classical separation techniques and by sterically controlled synthesis. For the purposes of this application, unless expressly noted to the contrary, a named amino acid shall be construed to include both the (D) or (L) stereoisomers. In the majority of cases, D- and L-amino acids have R- and S-absolute configurations, respectively.

The names of the natural amino acids are abbreviated herein in accordance with the recommendations of IUPAC-IUB.

“Small molecule” refers to a composition which has a molecular weight of less than about 2000 amu, or less than about 1000 amu, and even less than about 500 amu.

A “target” shall mean a site to which targeted constructs bind. A target may be either in vivo or in vitro. In certain embodiments, a target may be a tumor (e.g., tumors of the brain, lung (small cell and non-small cell), ovary, prostate, breast and colon as well as other carcinomas and sarcomas). In other embodiments, a target may be a site of infection (e.g., by bacteria, viruses (e.g., HIV, herpes, hepatitis) and pathogenic fuingi (Candida sp.). Certain target infectious organisms include those that are drug resistant (e.g., Enterobacteriaceae, Enterococcus, Haemophilus influenza, Mycobacterium tuberculosis, Neisseria gonorrhoeae, Plasmodium falciparum, Pseudomonas aeruginosa, Shigella dysenteriae, Staphylococcus aureus, Streptococcus pneumoniae). In still other embodiments, a target may refer to a molecular structure to which a targeting moiety binds, such as a hapten, epitope, receptor, dsDNA fragment, carbohydrate or enzyme. Additionally, a target may be a type of tissue, e.g., neuronal tissue, intestinal tissue, pancreatic tissue etc.

“Target cells”, which may serve as the target for the method of the present invention, include prokaryotes and eukaryotes, including yeasts, plant cells and animal cells. The present method may be used to modify cellular fuinction of living cells in vitro, i.e., in cell culture, or in vivo, in which the cells form part of or otherwise exist in plant tissue or animal tissue. Thus the cells may form, for example, the roots, stalks or leaves of growing plants and the present method may be performed on such plant cells in any manner which promotes contact of the targeted construct with the targeted cells. Alternatively, the target cells may form part of the tissue in an animal. Thus the target cells may include, for example, the cells lining the alimentary canal, such as the oral and pharyngeal mucosa, cells forming the villi of the small intestine, cells lining the large intestine, cells lining the respiratory system (nasal passages/lungs) of an animal (which may be contacted by inhalation of the subject invention), dermal/epidermal cells, cells of the vagina and rectum, cells of internal organs including cells of the placenta and the so-called blood/brain barrier, etc.

The term “targeting moiety” refers to any molecular structure which assists the construct in localizing to a particular target area, entering a target cell(s), and/or binding to a target receptor. For example, lipids (including cationic, neutral, and steroidal lipids, virosomes, and liposomes), antibodies, lectins, ligands, sugars, steroids, hormones, nutrients, and proteins may serve as targeting moieties.

A “patient,” “subject” or “host” to be treated by the subj ect method may mean either a human or non-human animal.

The termn “bioavailable” means that a compound the subject invention is in a formn that allows for it, or a portion of the amount administered, to be absorbed by, incorporated to, or otherwise physiologically available to a subject or patient to whom it is administered.

The phrases “parenteral administration” and “administered parenterally” are art-recognized terms, and include modes of administration other than enteral and topical administration, such as injections, and include, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is an art-recognized term which includes curing as well as ameliorating at least one symptom of any condition or disease. Diagnostic applications are also examples of “treating”.

The phrase “pharmaceutically acceptable” is art-recognized. In certain embodiments, the term includes compositions, subject coordination complexes and ligands, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The phrases “systemic administration,” “administered systemically,” “peripheral administration” and “administered peripherally” are art-recognized, and include the administration of a subject supplement, composition, therapeutic or other material other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of the therapeutic agent that produces some desired effect at a reasonable benefit/risk ratio applicable to any medical treatment. In certain embodiments, the term refers to that amount necessary or sufficient for diagnostic use of the subject compositions. One of ordinary skill in the art may empirically determine the effective amount of a particular compound without necessitating undue experimentation.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

Contemplated equivalents of the subject coordination complexes and other compositions described herein include such materials which otherwise correspond thereto, and which have the same general properties thereof, wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

II. General

A variety of sensors, and methods of using and making the same, are contemplated by the present invention. Examples of such sensors are set forth in Formulae 1 and 7. In addition, the components that make up such sensors, such as the ligand V—F, optionally tethered to a ligand (e.g., a macrocycle) of the subject coordination complexes, are also contemplated. In certain embodiments, the subject sensors react with an analyte of interest, optionally reversibly, with a concomitant change in the fluorescent properties of the resulting sensor complex as compared to the uncomplexed sensor. For example, upon exposure to an analyte, the fluorescence intensity of a sensor may increase. In certain embodiments, such sensors may be used to assay for small molecules, including without limitation, nitric oxide, carbon monoxide, carbon dioxide, dioxygen, dinitrogen, and cyanide. A variety of methods of preparing such sensors and their coordination complexes, of assaying for the binding activity of such sensors, and of using such compositions are also taught by the subject invention. A number of different sensors and ligands are contemplated for the subject coordination complexes, as set out in more detail below.

III Exemplary Sensors and Methods of Use Thereof.

III.a. Sensors Comprising Macrocyclic Ligands

In certain embodiments, the subject invention is directed to coordination complexes generally represented by the moiety of Formula 1: {M(MC)(V—F)}; wherein: MC represents a macrocycle that is capable of coordinating a metal ion through at least two Lewis basic atoms; M is a metal ion; V is a metal binding domain that is capable of forming a coordinate bond with M; and F represents a moiety which is capable of fluorescing. In certain embodiments, a coordination complex of Formula 1 may be charged. In certain embodiments, a coordination complex of Formula 1 may have additional components, such as other ligands, counter-ions, molecules of solvation and the like. In certain embodiments, V—F may be tethered to the macrocycle MC through a covalent tether. In certain embodiments, the macrocycle MC may be derivatized to enhance analyte binding, the reversibility of analyte binding, and other properties of the resulting coordination complex.

In certain embodiments, V—F in Formula 1 may be tethered to the MC through a covalent tether. Exemplary tether moieties are described in Section IIIe.

In certain embodiments, V—F in Formula 1 may not be coordinated to the metal ion of the coordination complex, but instead be associated with the metallomacrocycle in a fashion (e.g., through non-covalent interactions such as hydrogen bonding, hydrophobic interactions, etc.) that allows the fluorescence of F to change upon exposure to an analyte.

In certain embodiments, a sensor of Formula 1 may exist transiently in solution. For example, the complex in which V—F is coordinate to the metal ion may be in equilibrium with the form of the coordination complex in which V—F is not bound but in solution. This observation is true of many coordination complexes, so that any depiction of a coordination complex contained in this specification may give rise to other species in solution.

In certain embodiments, the sensors of the present invention are represented by Formula 1 and the attendant definitions, wherein the metal ion is a transition metal. In certain embodiments, the sensors of the present invention are represented by Formula 1 and the attendant definitions, wherein the metal ion may be selected from the group comprising cobalt, iron, zinc, vanadium, nickel, copper, chromium, manganese, and molybdenum. In certain embodiments, the sensors of the present invention are represented by Formula 1 and the attendant definitions, wherein the metal ion is cobalt. Other exemplary metal ions for use with the sensors of the present invention are described below in Section IIIc.

In certain embodiments, the sensors of the present invention are represented by Formula 1 and the attendant definitions, wherein the macrocycle represents a porphyrin or related macrocycle.

A number of different macrocycles may be used in the present invention, as will be known to one of skill in the art. Exemplary macrocycles include porphyrins, pthalocyanines, glyoximates, corroles, sapphyrins, salens, acens, crown ethers, azacrown ethers, cyclams, and the like.

Exemplary porphyrins include tetraphenylporphyrins, hemes, chlorophylls, chlorins, hemins, and corrins (some of which are understood to contain metal ions). Discussion of and examples of suitable macrocycles are provided in “Principles and Applications of Organotransition Metal Chemistry”, Collman, J. P., et al. 1987 University Science Books, CA. and “Inorganic Chemistry”, Huheey, J. E., et al. 4^{th} Ed. 1993, HarperCollins. Other macrocycles that may be used in the present invention are known to those of skill in the art.

In certain embodiments, the atoms of the macrocycle that are Lewis basic are heteroatoms such as nitrogen, oxygen, phosphorus, and sulfur. Because the Lewis basic groups function as the coordination site or sites for the metal ion, which in turn binds the ligand to be detected by the sensor, in certain embodiments, it may be preferable that the deformability of the electron shells of the Lewis basic groups and the metal ion be approximately similar. Such a relationship often results in a more stable coordination bond.

Any of the macrocyles used in the present invention be substituted in a manner that does not materially interfere with their use as a sensor hereunder. In certain embodiments, substitution of the macrocyle of a subject sensor may be used to modify the analyte specificity of the sensor, the solubility of the sensor, the reversibility of analyte binding, the fluorescence properties of the sensor and other physical and chemical properties of relevance to the present invention.

In certain embodiments, the sensors of the present invention comprise the generalized structure of Formula 2 and attendant definitions:

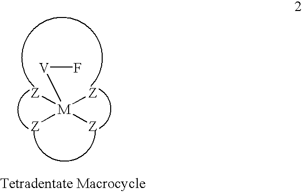

wherein:

tetradentate macrocycle is a macrocycle that coordinates a metal through four Z, wherein Z represents a Lewis basic atom;

M is a metal ion;

V is a metal binding domain; and

F is a fluorophore.

In certain embodiments, V—F may be tethered to the macrocycle through covalent bonds.

In certain other embodiments, the sensors of the present invention comprise a tetradentate macrocycle and the generalized structure of Formula 3 and attendant definitions:

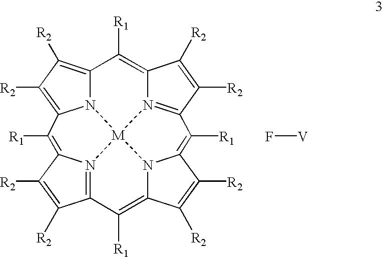

wherein:

M is a metal ion;

F is a fluorophore;

V is a metal binding domain;

R₁ optionally represents, independently for each occurrence, one or more substituents of the indicated pyrrole ring carbon that does not preclude coordination to a metal ion;

R₂ optionally represents, independently for each occurrence, one or more substituents of the indicated methene bridge that does not preclude coordination to a transition metal ion; and

V—F may optionally be bound to the ring structure through R₁ or R₂ via a covalent tether.

R₁ may be any one or more substituents at any of the indicated pyrrole ring carbon positions. In certain embodiments each R₁, independently, may be a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, amino, thio, lower alkoxy, lower alkylthio, lower alkylamino, nitro, phenoxy, benzyloxy, hydrogen, amine, hydroxyl, alkoxyl, carbonyl, acyl, formyl, sulfonyl and the like.

R₂ may be any one or more substituents at any of the methene carbon positions. In certain embodiments each R₂, independently, may be a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, amino, thio, lower alkoxy, lower alkylthio, lower alkylamino, nitro, phenoxy, benzyloxy, hydrogen, amine, hydroxyl, alkoxyl, carbonyl, acyl, formyl, sulfonyl and the like.

In certain other embodiments, F is comprised of a rhodafluor with the general structure in Formula 4 below:

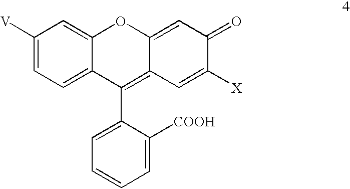

wherein,

V is a metal binding domain; and

X is any non-interfering substituent, preferably halogen, and most preferably chlorine.

In certain embodiments, the aromatic rings of the molecule of Formula 4 may have one or more non-interfering sub stituents, such as those sub stituents described for R₁, R₂, R₃ and R₄ of Formula 12 below.

Other exemplary V—F ligands for use with the sensors of the present invention are described below in Section IIId.

In other embodiments of the foregoing sensor, the sensor comprises the following structure of Formula 5 and attendant definitions:

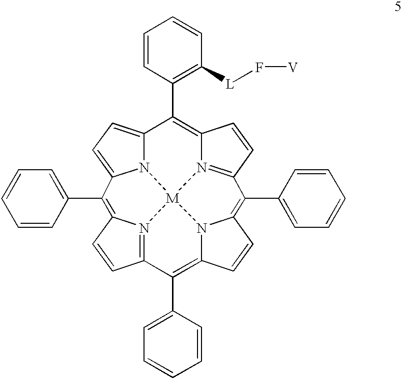

wherein L is a tether.

In still other embodiments, the sensors of the present invention comprise the generalized structure of Formula 6 and attendant definitions:

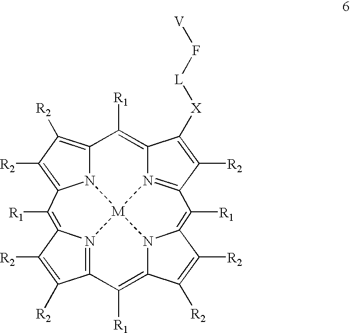

wherein X—L is the tether.

In certain embodiments of the invention, the subject macrocycle is at least approximately planar and a metal ion bound by such macrocycle will have axial coordination sites available to bind a fluorophore with a metal binding domain, leaving one available axial site. A ligand in that axial site trans to the bound fluorophore may be used to modify the specificity of the subject sensors, so that a particular sensor may selectively bind one analyte over others when one ligand is present the in trans axial position, and other analytes are favored when a different ligand is present in that site.

The design of a sensor for detecting a particular ligand will be possible by one of skill in the art, wherein issues such as selectivity, quantum yield, ease of synthesis and the like will be important criteria. Exemplary principles that may be used to design the subject sensors are presented in the Exemplification.

All of the foregoing coordination complexes of the present invention may further contain any one of the following: ligands in addition to a macrocycle and V—F, optionally tethered, capable of coordinating to the metal ion; counterions, waters of solvation, and other constituents commonly found in coordination compounds and know to those of skill in the art.

A number of different ligands capable of mono- and bidentate coordination may be used in the present invention, as will be known to one of skill in the art.

III.b. Sensors Comprising Bimetallic Ligands

In certain embodiments, the subject invention is directed to coordination complexes generally represented by the moiety of Formula 7: {Mₘ(W)ₙ(V—F)ₚ}; wherein independently for each occurrence: W represents a ligand which is capable of coordinating one or more metal ions through at least two Lewis basic atoms; M is a metal ion; V is a metal binding domain that is capable of forming a coordinate bond with M; F represents a moiety which is capable of fluorescing; m is at least 2, and n and p are each independently 1,2,3 or 4. In certain embodiments, a coordination complex of Formula 7 may be charged. In certain embodiments, the coordination complex of Formula 7 may have additional components, such as other ligands, counter-ions, molecules of solvation and the like. In certain embodiments, V—F may be tethered to W or another ligand of the coordination complex through a covalent tether. In certain embodiments, W and other ligands of the coordination complex may be derivatized to enhance analyte binding, the reversibility of analyte binding, and other properties of the resulting coordination complex.

In certain embodiments, V—F in Formula 7 may be tethered to W through a covalent tether. Exemplary tether moieties are described in Section IIIe.

In certain embodiments, V—F in Formula 7 may not be coordinated to the metal ion of the coordination complex, but instead be associated with the metal complex in a fashion (e.g., through non-covalent interactions such as hydrogen bonding, hydrophobic interactions, etc.) that allows the fluorescence of F to change upon exposure to an analyte.

In certain embodiments, a sensor of Formula 7 may exist transiently in solution. For example, the complex in which V—F is coordinate to the metal ion may be in equilibrium with the form of the coordination complex in which V—F is not bound but in solution. This observation is true of many coordination complexes, so that any depiction of a coordination complex contained in this specification may give rise to other species in solution.

In certain embodiments, the sensors of the present invention are represented by Formula 7 and the attendant definitions, wherein the metal ion is a transition metal. In certain embodiments, the sensors of the present invention are represented by Formula 7 and the attendant definitions, wherein the metal ion may be selected from the group comprising cobalt, iron, rhodium, ruthenium, vanadium, nickel, copper, chromium, manganese, and molybdenum, among other transition metals. In certain embodiments, the sensors of the present invention are represented by Formula 7 and the attendant definitions, wherein the metal ion is cobalt. Other exemplary metal ions for use with the sensors of the present invention are described below in Section IIIc.

In certain embodiments, the sensors of the present invention are represented by Formula 7 and the attendant definitions, wherein W represents a bidentate ligand.

In certain embodiments, the sensors of the present invention are represented by Formula 7 and the attendant definitions, wherein W represents a carboxylate ligand or sulfur-substituted derivative.

In certain embodiments, the atoms of the ligand that are Lewis basic are heteroatoms such as nitrogen, oxygen, phosphorus, and sulfur. Because the Lewis basic groups function as the coordination site or sites for the metal ion, which in turn binds the ligand to be detected by the sensor, in certain embodiments, it may be preferable that the deformability of the electron shells of the Lewis basic groups and the metal ion be approximately similar. Such a relationship often results in a more stable coordination bond.

Any of the ligands used in the present invention be substituted in a manner that does not materially interfere with their use as a sensor hereunder. In certain embodiments, substitution of the ligand of a subject sensor may be used to modify the analyte specificity of the sensor, the solubility of the sensor, the fluorescence properties of the sensor and other physical and chemical properties of relevance to the present invention.

In certain embodiments, the sensors of the present invention comprise the generalized structure of Formula 8 and attendant definitions:

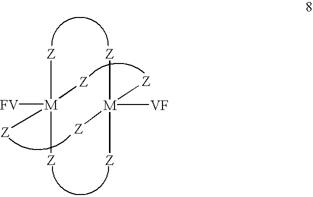

wherein, independently for each occurrence:

Z represents a Lewis basic atom;

M is a metal ion;

V is a metal binding domain; and

F is a fluorophore.

In certain embodiments, there will be a single V—F in Formula 8 (as opposed to two) and optionally a ligand in place of the other V—F.

In certain embodiments, the coordination complex of Formula 8 may be depicted with the structure of Formula 9 below. In certain embodiments, both species are present in a solution of the complex. Still other forms of the coordination complex (including those in which a ligand is no longer coordinated to a metal ion of the complex) may also be present in solution.

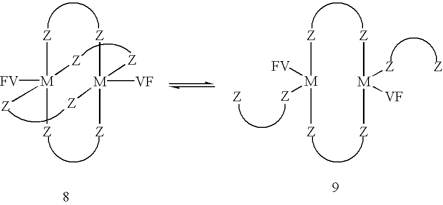

In certain embodiments, Z—Z is a carboxylate ligand, such that Z is O and the structure of Z—Z is (O—C(L)—O)⁻.

In certain embodiments, V—F may be tethered to Z—Z.

In certain other embodiments, the sensors of the present invention are bimetallic and comprise four carboxylate ligands and the generalized structure of Formula 10 and attendant definitions:

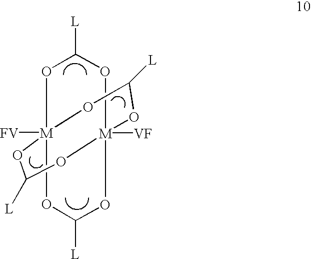

wherein, independently for each occurrence:

M is a metal ion;

F is a fluorophore;

V is a metal binding domain;

L is a carboxylate group substituent; and

V—F may optionally be bound to an L.

L may be any one or more substituents at any of the indicated carboxylate positions. In certain embodiments each L, independently, may be any linear or branched aliphatic, heteroaliphatic, aryl, heteroaryl, arylalkyl, heterocyclic or heteraromatic group. For example, L may be a methyl, ethyl, propyl, butyl, pentyl, hexyl, methoxyethyl, ethyxoyethyl, cyclopropyl, cyclobutyl, cyclopentyl, cyclohexyl, furyl, tetrahydrofuryl, phenyl, terphenyl, benzyl, phenylethyl, methoxyphenyl, napthyl, pyridyl, pyridazyl, pyrimidyl, piperidyl, piperazyl, pyrrolyl, pyrrolidyl, pyrazolyl, imidazolyl, thioalkyl, thiazolyl, thiopheneyl, thiophenyl, or silyl group, and the like. Any of the foregoing moieties may be optionally substituted.

In certain embodiments, there will be a single V—F in Formula 10 (as opposed to two) and optionally a ligand in place of the other V—F.

As for the coordination complex of Formula 8, as discussed above, Formula 10 may be depicted with the structure of Formula 11 below.

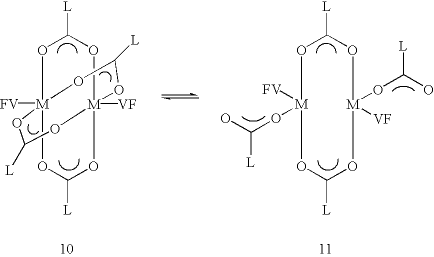

In certain embodiments, each L, independently, may comprise the generalized structure of Formula 12:

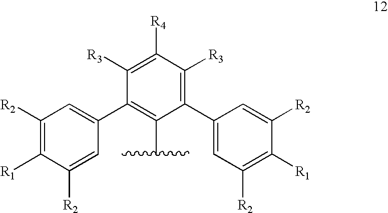

wherein: R₁, R₂, R₃ and R₄ optionally represents, independently for each occurrence, one or more substituents that does not preclude coordination to a metal ion.

In certain embodiments each Rₙ, independently, may be hydrogen, a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, amino, thio, lower alkoxy, lower alkylthio, lower alkylamino, nitro, phenoxy, benzyloxy, hydrogen, amine, hydroxyl, alkoxyl, carbonyl, acyl, formyl, sulfonyl and the like.

In certain other embodiments, F is comprised of a rhodafluor with the general structure in Formula 13 and 14 below:

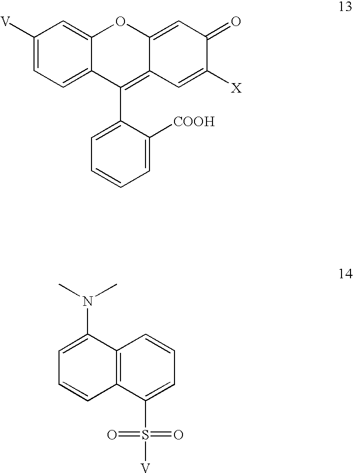

wherein,

V is a metal binding domain; and

X is any non-interfering substituent, preferably halogen, and most preferably chlorine.

In certain embodiments, the aromatic rings of the molecule of Formulae 13 and 14 may have one or more non-interfering substituents, such as those substituents described for R₁, R₂, R₃ and R₄ of Formula 12 above.

Other exemplary V—F ligands for use with the sensors of the present invention are described below in Section IIId.

In other embodiments of the foregoing sensor, the sensor comprises the following structure of Formula 15 and attendant definitions:

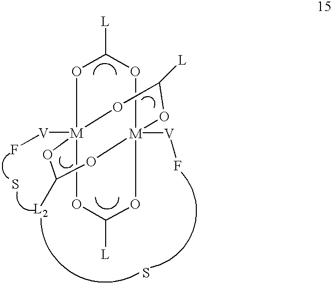

wherein L₂ is a tether. Exemplary tether moieties are described in Section IIIe.

In certain embodiments, L₂ comprises the following structure:

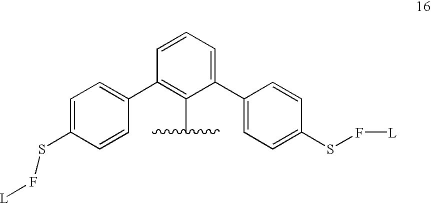

All of the foregoing coordination complexes of the present invention may further contain any one of the following: ligands in addition to a bridging ligand and V—F, optionally tethered, capable of coordinating to the metal ion; counterions, waters of solvation, and other constituents commonly found in coordination compounds and know to those of skill in the art.

A number of different ligands capable of mono- and bidentate coordination may be used in the present invention, as will be known to one of skill in the art.

III.c. Metal Atoms Comprised by the Subject Sensors

The metal atom comprised by the subject sensors may be selected from those that have usually at least four, five, six, seven coordination sites or more. In certain embodiments, the subject sensors may be capable of coordinating a wide range of metal ions, including light metals (Groups IA and IIA of the Periodic Table), transition metals (Groups IB-VIIIB of the Periodic Table), posttransition metals, metals of the lanthanide series and metals of the actinide series. In certain embodiments of the present invention, metal ions having unfilled d-shells will be preferred. In certain embodiments, transition metal ions from the first or second row will be preferred. In certain embodiments, transition metal ions from the third or fourth row will be preferred. A non-limiting list of metal ions which may be employed (including exemplary oxidation states for them) includes: Co³⁺, Cr³⁺, Hg²⁺, Pd²⁺, Pt²⁺, Pd⁴⁺, Pt⁴⁺, Rh³⁺,Rh²⁺, Ir³⁺, Ru³⁺, Ru²⁺, Co²⁺, Ni²⁺, Cu²⁺, Zn²⁺, Cd²⁺, Pb²⁺, Mn²⁺, Fe³⁺, Fe²⁺, Au³⁺, Au⁺, Ag⁺, Cu⁺, MoO₂²⁺, Ti³⁺, Ti⁴⁺, Bi³⁺, CH₃Hg⁺, Al³⁺, Ga³⁺, Ce³⁺, UO₂²⁺, Y⁺³, Eu, Gd and La³⁺.

III.d. Fluorophores with Metal Binding Domains for Use in the Subject Sensors

A number of different fluorophores having metal binding domains may be used in the present invention, e.g., as F in V—F, as will be known to one of skill in the art. Exemplary moieties that fluoresce include groups having an extensive delocalized electron system, eg. cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, indoaniline dyes, bis(S,O-dithiolene) complexes, and the like. Examples of suitable organic or metallated fluorophores may be found in “Topics in Applied Chemistry: Infrared absorbing dyes” Ed. M. Matsuoka, Plenum, NY 1990, “Topics in Applied Chemistry: The Chemistry and Application of Dyes”, Waring et al., Plenum, NY, 1990, “Handbook of Fluorescent Probes and Research Chemicals” Haugland, Molecular Probes Inc, 1996, DE-A-4445065, DE-A-4326466, JP-A-3/228046, Narayanan et al. J. Org. Chem. 60: 2391-2395 (1995), Lipowska et al. Heterocyclic Comm. 1: 427-430 (1995), Fabian et al. Chem. Rev. 92: 1197 (1992), W096/23525, Strekowska et al. J. Org. Chem. 57: 4578-4580 (1992), and WO96/17628, U.S. Pat. No. 6,051,207, and the Seventh Edition of the Handbook of Fluorescent Probes and Research Chemicals published by Molecular Probes, Inc. (http://www.probes.com/handbook/sections/0000.html) Particular examples of fluorophores which may be used include xylene cyanole, fluorescein, dansyl, rhodafluor, rhodamine, coumarin, acridine, resofurin, NBD, indocyanine green, DODCI, DTDCI, DOTCI, DDTCI and derivatives thereof.

In certain embodiments, the sensors of the present invention are represented by Formula 1 or 7 and the attendant definitions, wherein the fluorophore F is a dansyl, rhodafluor, rhodamine, coumarin, acridine, or resofurin derivative.

The fluorophores of the subject invention include a metal binding domain, V. V is intended to encompass numerous chemical moieties having a variety of structural, chemical and other characteristics capable of forming coordination bonds with a metal ion. The types of functional groups capable of forming coordinate complexes with metal ions are too numerous to categorize here, and are known to those of skill in the art. In certain embodiments, the atoms that are Lewis basic in V are heteroatoms such as nitrogen, oxygen, sulfur, and phosphorus.

Exemplary Lewis basic moieties which may be included in V include (assuming appropriate modification of them to allow for their incorporation into V and the subject fluorophores): amines (primary, secondary, and tertiary) and aromatic amines, amino groups, amido groups, nitro groups, nitroso groups, amino alcohols, nitrites, imino groups, isonitriles, cyanates, isocyanates, phosphates, phosphonates, phosphites, phosphines, phosphine oxides, phosphorothioates, phosphoramidates, phosphonamidites, hydroxyls, carbonyls (e.g., carboxyl, ester and formyl groups), aldehydes, ketones, ethers, carbamoyl groups, thiols, sulfides, thiocarbonyls (e.g., thiolcarboxyl, thiolester and thiolformyl groups), thioethers, mercaptans, sulfonic acids, sulfoxides, sulfates, sulfonates, sulfones, sulfonamides, sulfamoyls and sulfinyls.

Illustrative of suitable V include those chemical moieties containing at least one Lewis basic nitrogen, sulfur, phosphorous or oxygen atom or a combination of such nitrogen, sulfur, phosphorous and oxygen atoms. The carbon atoms of such moiety may be part of an aliphatic, cycloaliphatic or aromatic moiety. In addition to the organic Lewis base functionality, such moieties may also contain other atoms and/or groups as substituents, such as alkyl, aryl and halogen substituents.

Further examples of Lewis base fanctionalities suitable for use in V include the following chemical moieties (assuming appropriate modification of them to allow for their incorporation into V and the subject fluorescein or dansyl based ligands): amines, particularly alkylamines and arylamines, including methylamine, diphenylamine, trimethylamine, triethylamine, N,N-dimethylaniline, methyldiphenylaniline, pyridine, aniline, morpholine, N-methylmorpholine, pyrrolidine, N-methylpyrrolidine, piperidine, N-methylpiperidine, piperazine, cyclohexylamine, n-butylamine, dimethyloxazoline, imidazole, N-methylimidazole, N,N-dimethylethanolamine, N,N-diethylethanolimine, N,N-dipropylethanolamine, N,N-dibutylethanolamine, N,N-dimethylisopropanolamine, N,N-diethylisopropanolamine, N,N-dipropylisopropanolamine, N,N-dibutylisopropanolamine, N-methyldiethanolamine, N-ethyldiethanolamine, N-propyldiethanolamine, N-butyldiethanolamine, N-methyldiisopropanolamine, N-ethyldiisopropanolamine, N-propyldiisopropanolamine, N-butyldiisopropanolamine, triethylamine, triisopropanolamine, tri-s-butanolamine and the like; amides, such as N,N-dimethylformamide, N,N-dimethylacetamide, N-methylpyrrolidone, hexamethylphosphoric acid triamide and the like; sulfoxide compounds, such as dimethylsulfoxide and the like; ethers such as dimethyl ether, diethyl ether, tetrahydrofuran, dioxane and the like; thioethers such as dimethylsulfide, diethyl thioether, tetrahydrothiophene and the like; esters of phosphoric acid, such as trimethyl phosphate, triethylphosphate, tributyl phosphate and the like; esters of boric acid, such as trimethyl borate and the like; esters of carboxylic acids, such as ethyl acetate, butyl acetate, ethyl benzoate and the like; esters of carbonic acid, such as ethylene carbonate and the like; phosphines including di- and trialkylphosphines, such as tributylphosphine, triethylphosphine, triphenylphosphine, diphenylphosphine and the like; and monohydroxylic and polyhydroxylicalcohols of from 1 to 30 carbon atoms such as methyl alcohol, ethyl alcohol, n-propyl alcohol, isopropyl alcohol, n-butyl alcohol, isobutyl alcohol, tert-butyl alcohol, n-pentyl alcohol, isopentyl alcohol, 2-methyl-1-butyl alcohol, 2-methyl-2-butyl alcohol, n-hexyl alcohol, n-heptyl alcohol, n-octyl alcohol, isooctyl alcohol, 2-ethylhexyl alcohol, n-nonyl alcohol, n-decyl alcohol, 1,5-pentanediol, 1,6-hexanediol, allyl alcohol, crotyl alcohol, 3-hexene-1-ol, citronellol, cyclopentanol, cyclohexanol, salicyl alcohol, benzyl alcohol, phenethyl alcohol, cinnamyl alcohol, and the like; and heterocyclic compounds, including pyridine and the like.

Other suitable structural moieties that may be included in V include the following Lewis base functionalities: arsine, stilbines, thioethers, selenoethers, teluroethers, thioketones, imines, phosphinimine, pyridines, pyrazoles, imidazoles, furans, oxazoles, oxazolines, thiophenes, thiazoles, isoxazoles, isothrazoles, amides, alkoxy, aryoxy, selenol, tellurol, siloxy, pyrazoylborates, carboxylate, acyl, amidates, triflates, thiocarboxylate and the like.

Other suitable ligand fragments for use in V include structural moieties that are bidentate ligands, including diimines, pyridylimines, diamines, imineamines, iminethioether, iminephosphines, bisoxazoline, bisphosphineimines, diphosphines, phosphineamine, salen and other alkoxy imine ligands, amidoamines, imidothioether fragments and alkoxyamide fragments, and combinations of the above ligands.

Still other suitable fragments for use in V include ligand fragments that are tridentate ligands, including 2,5-diiminopyridyl ligands, tripyridyl moieties, triimidazoyl moieties, tris pyrazoyl moieties, and combinations of the above ligands.

Other suitable ligand fragments may consist of amino acids or be formed of oligopeptides and the like.

Because the Lewis basic groups function as the coordination site or sites for the metal cation, in certain embodiments, it may be preferable that the deformability of the electron shells of the Lewis basic groups and the metal cations be approximately similar. Such a relationship often results in a more stable coordination bond. For instance, sulfur groups may be desirable as the Lewis basic groups when the metal cation is a heavy metal. Some examples include the oligopeptides such as glutathione and cysteine, mercapto ethanol amine, dithiothreitol, amines and peptides containing sulfur and the like. Nitrogen containing groups may be employed as the Lewis basic groups when smaller metal ions are the metal. Alternatively, for those applications in which a less stable coordination bond is desired, it may be desirable that the deformability be dissimilar.

In certain embodiments, V may by comprised of a piperazine or piperidine moiety.

In certain embodiments, it may be the case that what is commonly known as a fluorophore to one of skill in the art contains a V, or metal binding domain, without any modifications to the fluorophore that may be used in the present invention. In other embodiments, a fluorophore is synthetically modified to incorporate a metal binding domain.

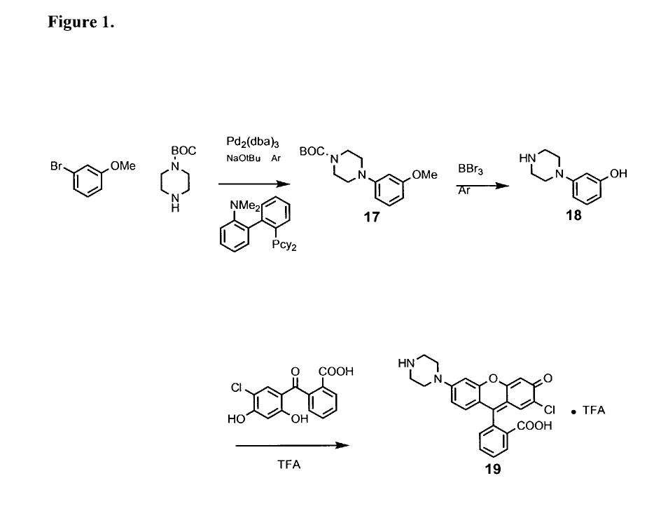

FIG. 1 depicts the synthesis of one V—F contemplated by the invention, Rhodapip, containing a rhodafluor moiety as F and a piperidine-like moiety as V. Another V—F, dansylpiperazine, has been reported in Saavedra, J. E. et al. J. Org. Chem. 1999, 64, 5124. As shown in the synthetic protocol, any number of metal binding groups V may be synthesized as part of V—F by condensing the appropriate substituted phenol with the diphenyl ketone shown in FIG. 1 to give a V—F molecule containing a rhodafluor moiety as F and a different V based on the substituted phenol used. Examples of such other metal binding domains are presented in U.S. Ser. No. 60/284,700 (filed Apr. 17, 2001), which is incorporated by reference into this application in its entirety. Such substituted phenols may be used to prepare bidentate, tridentate and other multidentate ligands in addition to the monodentate ligand found in Rhodapip. In addition, substitution other than with —Cl of the rhodafluor moiety may be achieved by using a different starting material in place of 2′-carboxy-5-chloro-2,4-dihydroxybenzophenone shown in FIG. 1.

III.e. Tether Moieties for Use in the Subject Sensors

A number of different tether moieties may be used in the subject inventions, as will be known to one of skill in the art. In certain embodiments, a tether is an organic moiety, such as a divalent branched or straight chain or cyclic aliphatic group or divalent aryl group, with in certain embodiments, from 1 to about 20 carbon atoms. In certain embodiments, a tether represents a moiety between about 2 and 20 atoms selected from carbon, oxygen, sulfur, and nitrogen, wherein at least 60% of the atoms are carbon.

In certain embodiments, a tether may be an alkylene group, such as methylene, ethylene, 1,2-dimethylethylene, n-propylene, isopropylene, 2,2-dimethylpropylene, n-pentylene, n-hexylene, n-heptylene; an alkenylene group such as ethenylene, propenylene, 2-(3-propenyl)-dodecylene; and an alkynylene group such as ethynylene, proynylene and the like. Other examples of tethers may ethylene glycol, propylene glycol or oligomers thereof and the like.

Further, a tether may be a cycloaliphatic group, such as cyclopentylene, 2-methylcyclopentylene, cyclohexylene, cyclohexylenedimethylene, cyclohexenylene and the like. A tether may also be a divalent aryl group, such as phenylene, benzylene, naphthalene, phenanthrenylene and the like. Further, a tether may be a divalent heterocyclic group, such as pyrrolylene, furanylene, thiophenylene, alkylyene-pyrrolylene-alkylene, pyridinylene, pyrimidinylene and the like.

The foregoing, as with all other moieties described herein, may be substituted with a non-interfering substituent, for example, as described above for R₁, R₂, R₃ and R₄ of Formula 12.

IV. Fluorescence Assays

A variety of different analytes may be used in the present invention. For example, analytes that are relatively sterically unhindered and monodentate ligands may be detected using the teachings of the present invention, including for example, nitric oxide, carbon monoxide, carbon dioxide, dioxygen, dinitrogen, and cyanide. Other suitable analytes will be known to those of skill in the art.

(1) Instrumentation

Fluorescence of a sensor provided by the present invention may be detected by essentially any suitable fluorescence detection device. Such devices are typically comprised of a light source for excitation of the fluorophore and a sensor for detecting emitted light. In addition, fluorescence detection devices typically contain a means for controlling the wavelength of the excitation light and a means for controlling the wavelength of light detected by the sensor. Such means for controlling wavelengths are referred to generically as filters and can include diffraction gratings, dichroic mirrors, or filters. Examples of suitable devices include fluorimeters, spectrofluorimeters and fluorescence microscopes. Many such devices are commercially available from companies such as Hitachi, Nikon or Molecular Dynamics. In certain embodiments, the device is coupled to a signal amplifier and a computer for data processing.

(2) General Aspects

In general, assays using sensors provided by the present invention involve contacting a sample with such a sensor and measuring fluorescence. The presence of a ligand that interacts with the sensor may alter fluorescence of the sensor in many different ways. Essentially any change in fluorescence caused by the ligand may be used to determine the presence of the ligand and, optionally, the concentration of the ligand in the sample.

The change may take one or more of several forms, including a change in excitation or emission spectra, or a change in the intensity of the fluorescence and/or quantum yield. These changes may be either in the positive or negative direction and may be of a range of magnitudes, which preferably will be detectable as described below.

The excitation spectrum is the wavelengths of light capable of causing the sensor to fluoresce. To determine the excitation spectrum for a sensor in a sample, different wavelengths of light are tested sequentially for their abilities to excite the sample. For each excitation wavelength tested, emitted light is measured. Emitted light may be measured across an interval of wavelengths (for example, from 450 to 700 nm) or emitted light may be measured as a total of all light with wavelengths above a certain threshold (for example, wavelengths greater than 500 nm). A profile is produced of the emitted light produced in response to each tested excitation wavelength, and the point of maximum emitted light can be referred to as the maximum excitation wavelength. A change in this maximum excitation wavelength, or a change in the shape of the profile caused by ligand in a sample may be used as the basis for determining the presence, and optionally, the concentration of metal in the sample. Alternatively, the emission spectrum may be determined by examining the spectra of emitted light in response to excitation with a particular wavelength (or interval of wavelengths). A profile of emissions at different wavelengths is created and the wavelength at which emission is maximal is called the maximum emission wavelength. Changes in the maximum emission wavelength or the shape of the profile that are caused by the presence of a ligand in a sample may be used to determine the presence or concentration of the ligand in the sample. Changes in excitation or emission spectra may be measured as ratios of two wavelengths. A range of changes are possible, from about a few nms to 5, 10, 15, 25, 50, 75 100 or more nms.

Quantum yield may be obtained by comparison of the integrated area of the corrected emission spectrum of the sample with that of a reference solution. A preferred reference solution is a solution of fluorescein in 0.1 N NaOH, quantum efficiency 0.95. The concentration of the reference is adjusted to match the absorbance of the test sample. The quantum yields may be calculated using the following equation.

$${Φ}_{sample} = {Φ}_{standard} \times \frac{\int {emission}_{sample}}{\int {emission}_{standard}} \times \frac{{Abs}_{standard}}{{Abs}_{sample}}$$

A change in quantum yield caused by a ligand may be used as the basis for detecting the presence of the ligand in a sample and may optionally be used to determine the concentration of the ligand. A range of changes are possible in the subject invention. For example, the difference in the quantum yield for a subject sensor in the presence of a ligand may be about 10%, 25%, 50%, 75% the quantum yield, or it may be 2, 3, 5, 10, 100, 200, 1000, 10000 times greater or more. The same values may be used to describe changes observed in intensity in such the subject assays.

It is expected that some samples will contain compounds that compete with the sensor for the ligand. In such cases, the fluorescence measurement will reflect this competition. In one variation, the fluorescence may be used to determine the presence or concentration of one or more such ligand-competing compounds in a sample.

(3) In vitro Assays

In one variation, the presence of a ligand in a sample is detected by contacting the sample with a sensor that is sensitive to the presence of the ligand. The fluorescence of the solution is then determined using one of the above-described devices, preferably a spectrofluorimeter. Optionally, the fluorescence of the solution may be compared against a set of standard solutions containing known quantities of the ligand. Comparison to standards may be used to calculate the concentration of the analyte, i.e., the ligand.

The ligand may be any substance described above. The concentration of the ligand may change over time and the fluorescent signal of the sensor may serve to monitor those changes. For example, the particular form of the ligand that interacts with the sensor may be produced or consumed by a reaction occurring in the solution, in which case the fluorescence signal may be used to monitor reaction kinetics.

In certain embodiments, the sample is a biological fluid, lysate, homogenate or extract. The sample may also be an environmental sample such as a water sample, soil sample, soil leachate or sediment sample. The sample may be a biochemical reaction mixture containing at least one protein capable of binding to or altering a metal. Samples may have a pH of about 5, 6, 7, 8, 9, 10, 11, 12 or higher.

(4) In vivo Assays

In another variation, the presence of a ligand in a biological sample may be determined using a fluorescence microscope and the subject sensors. The biological sample is contacted with the sensor and fluorescence is visualized using appropriate magnification, excitation wavelengths and emission wavelengths. In order to observe co-localization of multiple analytes, the sample may be contacted with multiple sensors simultaneously. In certain embodiments the multiple sensors differ in their emission and/or excitation wavelengths.

Biological samples may include bacterial or eukaryotic cells, tissue samples, lysates, or fluids from a living organism. In certain embodiments, the eukaryotic cells are nerve cells, particularly glutamate neurons. In other embodiments, the eukaryotic cells are neurons with mossy fiber terminals isolated from the hippocampus. Tissue samples are preferably sections of the peripheral or central nervous systems, and in particular, sections of the hippocampus containing mossy fiber terminals. It is also anticipated that the detection of a ligand in a cell may include detection of the ligand in subcellular or extracellular compartments or organelles. Such subcellular organelles and compartments include: Golgi networks and vesicles, pre-synaptic vesicles, lysosomes, vacuoles, nuclei, chromatin, mitochondria, chloroplasts, endoplasmic reticulum, coated vesicles (including clathrin coated vesicles), caveolae, periplasmic space and extracellular matrices.

(5) Assays Using a Nitric Oxide Sensor of the Present Invention

In certain embodiments of the above assays, the sensor is an NO sensor and the ligand is NO. The solution or biological sample is contacted with an NO sensor, and fluorescence of the sensor is excited by light with an appropriate wavelength for the fluorophore of the sensor as known to one of skill in the art. Light emitted by the sensor is detected by detecting light of the expected emission wavelength of the fluorophore of the sensor as known to one of skill in the art.

Exemplification

General Experimental Considerations. Tetrahydrofuran (THF), diethyl ether and pentane were purified by passage through columns of alumina under N₂. Pangborn, A. B. et al. Organometallics 1996, 15, 1518-1520. Dichloromethane (CH₂Cl₂), chlorobenzene and triethylamine (Et₃N) were distilled from CaH₂ under N₂. 2-(Tosyloxy)tropone, H(i-Pr)₂ATI, 2,2′-(Pentamethylenediamino)di-2,4,6-cycloheptatrien-1-one (“4-dimer-tropolone”), [Co(CH₃CN)₄](PF₆)₂, and HBAr'₄.(Et₂O)₂ were prepared as described in the literature. Doering, W. v. E.; Hiskey, C. F. J. Am. Chem. Soc. 1952, 74, 5688; Dias, H. V. R.; Jin, W.; Ratcliff, R. E. Inorg. Chem. 1995, 34, 6100-6105; Goldstein, A. S.; Drago, R. S. Inorg. Chem. 1991, 30, 4506-4510; Brookhart, M.; Grant, B.; Volpe, A. F. J. Organometallics 1992, 11, 3920-3922; Zask, A. et al. Inorg. Chem. 1986, 25, 3400-3406. Nitric oxide (Matheson, 99%) and ¹⁵NO (Aldrich, 99%) were purified of higher nitrogen oxides by passage through a column of NaOH pellets and a mercury bubbler and kept over mercury in gas storage bulbs. Analysis by GC of the NO used in the experiments revealed no contaminants, such as NO₂ or N₂O, at the limit of the thermal conductivity detector, about 30 nM. All other reagents were obtained commercially and not further purified. Silica gel 60 (230-400 mesh, EM Science) or activated basic alumina (150 mesh, Brockmann I) was used for column chromatography. UV-visible spectra were recorded on a Hewlett Packard 8435 spectrophotometer. Standard IR spectra were recorded on a Bio Rad FTS-135 instrument; solid samples were prepared as pressed KBr discs and solution samples were prepared in an airtight Graseby-Specac solution cell with CaF₂ windows. In situ IR sample monitoring was performed with a ReactIR 1000 from ASI Applied Systems equipped with a 1-in diameter, 30-reflection silicon ATR (SiComp) probe optimized for maximum sensitivity. Reaction protocols are as described previously. Franz, K. J.; Lippard, S. J. J. Am. Chem. Soc. 1999, 121, 10504-10512. Fluorescence emission spectra were recorded at 25±1° C. on a Hitachi F-3010 fluorescence spectrophotometer. Mass spectra were determined in a 3-nitrobenzyl alcohol matrix with a Finnegan 4000 mass spectrometer using 70-eV impact ionization. Melting points were measured on a Thomas Hoover capillary melting point apparatus. NMR spectra were recorded on a Bruker AC 250 or Varian Mercury 300 MHz spectrometer at ambient probe temperature and referenced to the internal ¹H and ¹³C solvent peak.

### EXAMPLE 1

### Nitric Oxide Sensor Comprised of Cobalt(II)Tetraphenylporphyrin and Rhodapip

Synthesis of Rhodapip. The generalized method of preparing Rhodapip and other like ligands is described in U.S. Ser. No. 10/124,742 (filed Apr. 17, 2002). The synthetic route to Rhodapip, a rhodafluor containing a piperidine-like moiety is shown in FIG. 1.

### 1. Synthesis of 4-(3-Methoxyphenyl)-piperazine-1-carboxylic acid-t-butul ester (17)

Under an atmosphere of Ar a Schlenk flask was charged with NaOtBu (1.44 g, 15.0 mmol), tris(dibenzylideneacetone)dipalladium(0) (99 mg, 0.108 mmol), t-butyl 1-piperazine-carboxylate (2.00 g, 10.7 mmol), and 2-dicyclohexylphosphino-2′-(N,N-dimethylamino)biphenyl (254 mg, 0.64 mmol) in 30 mL dry toluene. The suspension was heated to 60° C. in an oil bath, 3-bromoanisole (1.16 ml, 9.12 mmol) was added via syringe, and the reaction was stirred for 2 h. The reaction was allowed to cool, was filtered through a medium frit, and the solvent was removed by rotary evaporation. Column chromatography on silica gel (17:3 hexanes/EtOAc) affords 17 as an off white solid (2.53 g, 95%). 1H NMR (400 MHz, CD2Cl2): d 7.15 (1H, t, J=8.1 Hz), 6.52 (1H, dd, J=8.2, 1.8 Hz), 6.44-6.40 (2H, m), 3.76 (3H, s), 3.53 (4H, t, J=5.1 Hz), 3.10 (4H, t, J=5.2 Hz), 1.45 (9H, s). IR (KBr, cm−1): 3441, 3011 2984, 2974, 2922, 2872, 2840, 1697, 1608, 1594, 1499, 1456, 1419, 1381, 1316, 1259, 1246, 1202, 1161, 1131, 1070, 1037, 995, 945, 863, 820, 762, 684, 640, 587, 566, 534, 512.

### 2. Synthesis of 1-(3-Hydroxyphenyl)piperazine (18)

Under an atmosphere of Ar, neat BBr3 (16.1 mL, 170 mmol) was transferred via syringe to a solution of 17 (2.50 g, 8.55 mmol) in 150 mL CH2Cl2. After stirring for 3 d under a positive pressure of Ar, the reaction was cooled in a dry-ice/acetone bath and quenched with 50 mL MeOH. The reaction mixture was poured into 300 mL of water and allowed to boil for 45 min. After cooling to room temperature, the aqueous solution was adjusted to pH 9 with NaOH resulting in formation of a cloudy suspension. This suspension was extracted with CH2Cl2 (3×75 mL), dried with MgSO₄, and the solvent was removed by rotary evaporation giving 18 as an off white solid (986 mg, 65%). 1H NMR (400 MHz CD3OD): d 7.04 (1H, t, J=8.1 Hz), 6.46 (1H, dd, J=8.1, 1.9Hz), 6.40 (1H, t, J=2.2Hz), 6.30 (1H, dd, J=8.0, 1.6Hz), 3.08 (4H, t, J=4.8 Hz), 2.95 (4H, t, J=4.7 Hz). 13C NMR (100 MHz, CD3OD): d 159.4, 154.8, 130.9, 109.3, 108.5, 104.8, 51.4, 46.6. IR (KBr, cm−1): 3064, 2974, 2959, 2938, 2922, 2857, 2845, 2679, 2580, 1597, 1503, 1454, 1385, 1359, 1320, 1276, 1241, 1201, 1181, 1164, 1132, 1104, 1068, 1031, 996, 877, 867, 841, 819, 767, 710, 687, 535, 498.

### 3. Synthesis of Rhodapip TFA (19)

To a solution of 18 (200 mg, 1.12 mmol) in 10 mL TFA, 2′-carboxy-5-chloro-2,4-dihydroxybenxophenone (1.314 g, 4.50 mmol) was added and heated to reflux for 3 d. The crude product was isolated by rotary evaporation of the TFA. Column chromatography on silica gel (100% Acetone, 100% MeOH) yielded clean Rhodapip (19) as a bright red solid (435 mg, 70.6%). X-ray quality blood red plates of Rhodapip were prepared by slow crystallization from an aqueous solution containing 100 mM KCl, and 50 mM PIPES at pH 7.0. MS-ESI (m/z): [M+H]+Calcd. for C24H19O4N2Cl, 435.1106; found 435.1100. 1H NMR (400 MHz CD₃CN): δ8.71 (s), 7.97 (1H, d, J=9.8 Hz), 7.75-7.66 (2H, m), 7.16 (1H, d, J=7.3 Hz), 6.96 (1H, s), 6.75-6.74 (2H, m), 6.68-6.63 (2H, m), 3.48 (4H, t, J=5.5 Hz), 3.28 (4H, t, J=5.6 Hz). The chemical shift at 8.71, the integration for which indicates 1.5 hydrogens, attributable to trifluoroacetic acid. The Rhodapip sample has not yet been purified after synthesis, and is believed to be a trifluoroacetate salt with one or both of the nitrogens protonated. FT-IR (KBr, cm−1): 3429, 3013, 3240, 1761, 1678, 1633, 1611, 1583, 1482,1428, 1386, 1254, 1200, 1130, 1037, 1012, 975, 874, 835, 797, 761, 721, 699, 611, 595, 543, 511, 480. The bands at 1678, 1200, and 1130 appear to be from trifluoroacetate or trifluoroacetic acid. HRMS (ESI(+)) Calcd. [M+H] 435.1106, found 435.1100

Cobalt(II)tetraphenylporphyrin (CoTPP) was obtained from a commercial source. 2′-carboxy-5-chloro-2,4-dihydroxybenzophenone was prepared as previously described. (Smith, G. A.; Metcalfe, J. C.; Clarke, S. D J. Chem. Soc. Perkin Trans 2. 1993, 1195.)

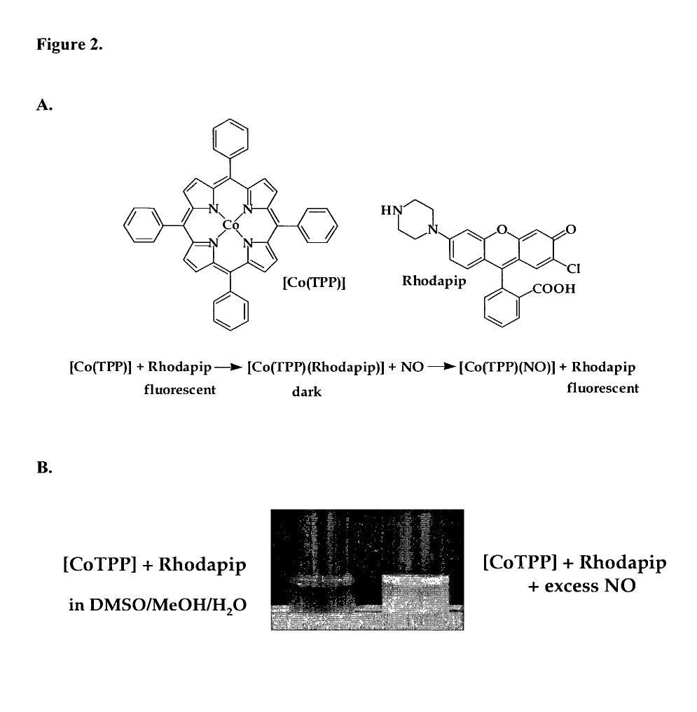

Detection of Nitric Oxide with Rhodapip and Co(TPP) Fluorescent Sensor. The structures of Rhodapip and Co(TPP) are shown in FIG. 2a. The free Rhodapip ligand exhibits fluorescence. Without intending to limit the invention in any way, as shown in the formula in FIG. 2a, it is believed that complexation of Rhodapip by Co(TPP) in the absence of nitric oxide should result in quenching of the fluorescence (FIG. 2a), whereupon addition of nitric oxide to the Rhodapip/Co(TPP) mixture should cause the Rhodapip ligand to be displaced, resulting in free Rhodapip and an increase in fluorescence (FIG. 2a). The ability of the Rhodapip/Co(TPP) system to act as a nitric oxide sensor was tested. A mixture of Co(TPP) and Rhodapip in DMSO/methanol/water exhibited little to no fluorescence (FIG. 2b, left). After exposure to nitric oxide, an increase in fluorescence was observed (FIG. 2b, right). The exact reaction that occurs in this experiment has not yet been determined.

### EXAMPLE 2

### Nitric Oxide Sensor Comprised of [Co₂(O₂CAr^{Tol})₄(dansylpiperazine)₂]

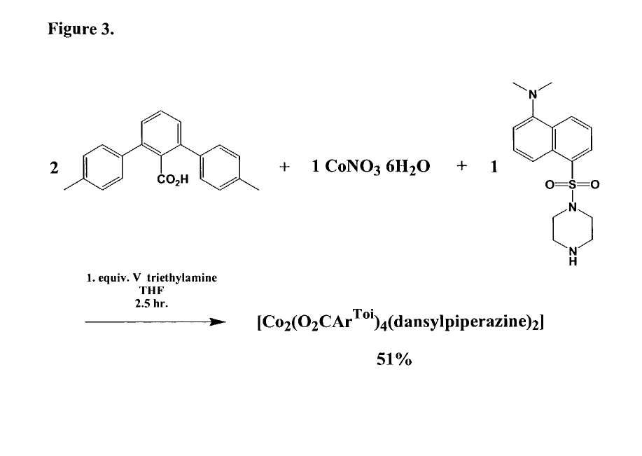

Synthesis of [Co₂(μ-O₂CAr^{Tol})₄(dansylpiperazine)₂]. The synthetic route to [Co₂(μ-O₂CAr^{Tol})₄(dansylpiperazine)₂] is shown in FIG. 3. Danzylpiperazine was prepared using the method of Saavedra, J. E. et al. J. Org. Chem. 1999, 64, 5124. The carboxylate ligands were prepared using methods as previously described. (Du, C.-J. F., et al. J. Org. Chem. 1986, 51, 3162; Saednya, A.; Hart, H. Synthesis 1996, 1455; and Chen, C.-T.; Siegel, J. S. J. Am. Chem. Soc. 1994, 116, 5959) X-ray quality crystals were grown by vapor diffusion of diethyl ether into methylene chloride to confirm the stoichiometry and structure shown. FT-IR (KBr, cm−1): 3435, 3247, 3048, 3019, 2984, 2939, 2920, 2863, 2786, 1614, 1585, 1512, 1449, 1403,1384, 1345, 1329, 1165, 1149, 1107, 1062, 1022, 932, 844, 814, 797, 707, 618, 584, 569, 527, 487.

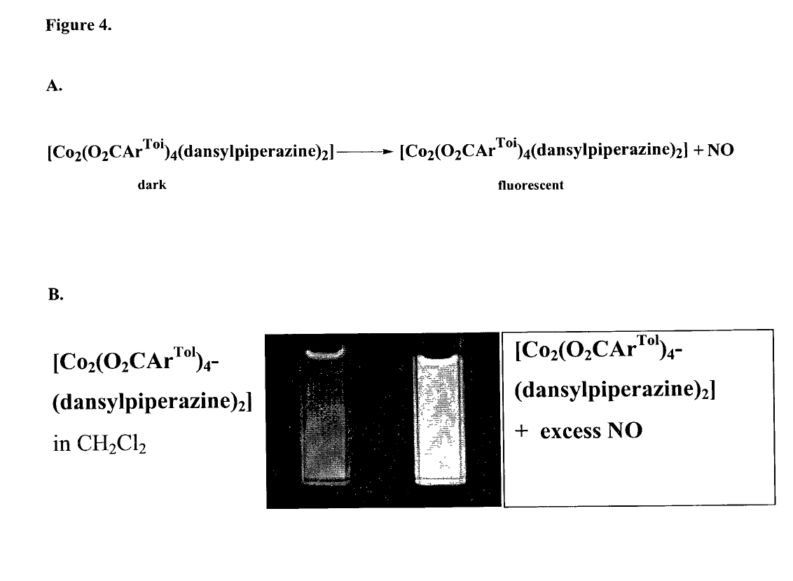

Detection of Nitric Oxide with the [Co₂(O₂CAr^{Tol})₄(dansylpiperazine)₂Fluorescent Sensor. It is believed that in solution [Co₂(O₂CAr^{Tol})₄(dansylpiperazine)₂] is in equilibrium between two species: the [Co₂(μ-O₂CAr^{Tol})₄(dansylpiperazine)₂] (paddle-wheel) isomer and the [Co₂(μ-O₂CAr^{Tol})₂(O₂CAr^{Tol})₂(dansylpiperazine)₂] (windmill) isomer. Without intending to limit the invention in any way, as shown in the formula in FIG. 4a, it is believed that complexation of dansylpiperazine in [Co₂(O₂CAr^{Tol})₄(dansylpiperazine)₂] in the absence of nitric oxide should result in quenching of the fluorescence (FIG. 4a), whereupon addition of nitric oxide to the complex causes the dansylpiperazine ligand to be displaced, resulting in free dansylpiperazine and an increase in fluorescence (FIG. 4a). The ability of the [Co₂(O₂CAr^{Tol})₄(dansylpiperazine)₂] system to act as a nitric oxide sensor was tested. A solution of [Co₂(O₂CAr^{Tol})₄(dansylpiperazine)₂] in dichloromethane exhibited little to no fluorescence (FIG. 4b, left). After exposure to nitric oxide, an increase in fluorescence was observed (FIG. 4b, right). The exact reaction that occurs in this experiment has not yet been determined. Without intending to limit the invention in any way, some preliminary evidence suggests that, as opposed to a simple reaction of dansylpiperazine by NO at the axial coordination site of one or more of the cobalt ions, the cobalt ions may be reduced to Co(I) upon exposure to NO, both mono- and bi-metallic species may be formed, and the dansylpiperazine may be covalently modified. In any case, it appears that upon dissociation from the cobalt ion upon exposure to NO, dansylpiperazine exhibits increased fluorescence upon excitation.

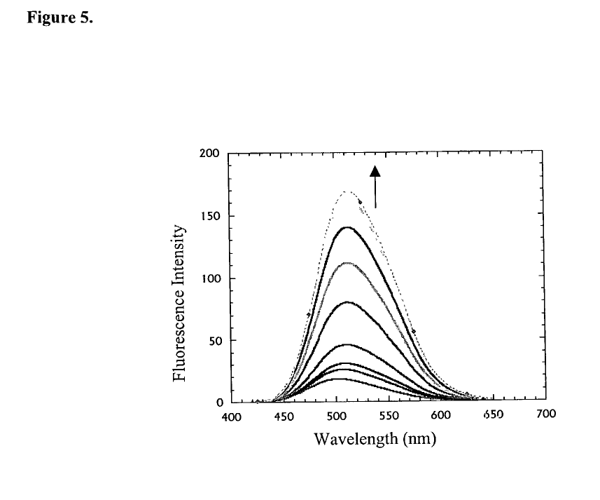

The fluorescence response of [Co₂(O₂CAr^{Tol})₄(dansylpiperazine)₂] after exposure to excess NO in CH₂Cl₂ was measured spectroscopically. Solutions were excited at 350 nm and the emission spectrum recorded. The peak emission intensity is at 503 nm before exposure NO, but shifts to 513 nm after exposure to NO. Emission spectra, shown in FIG. 5, were recorded at 0, 1, 5, 10, 20, 30, 40, 50, and 60 minutes post exposure to NO. A fluorescence increase of 9.6-fold was observed after 60 minutes.

### INCORPORATION BY REFERENCE

All publications and patents mentioned herein, including those items listed below, are hereby incorporated by reference in their entirety as if each individual publication or patent was specifically and individually incorporated by reference. In case of conflict, the present application, including any definitions herein, will control.

Advanced Inorganic Chemistry, 5^{th} ed.; Wiley-Interscience: New York, 1988.

Anderegg et al. Helv. Chim. Acta 1977, 60: 123-140

Atar, et al. J. Biol. Chem. 1995, 270: 2473-2477

Barker, S. L. R. et al. Anal. Chem. 1998, 70, 971-976.

Barker, S. L. R., et al Anal. Chem. 1998, 70, 4901-4906.

Barker, S. L. R. et al. Anal. Chem. 1999, 71, 1767-1772.

Barker, S. L. R. et al. Anal. Chem. 1999, 71, 2071-2075.

Bätz, M. et al. Angew. Chem. Int. Ed. Engl. 1997, 36, 1501-1503.

Belgodere et al. Heterocycles 1985, 23, 349-354.

Bergonzi, R. et al. Coord. Chem. Rev. 1998, 170, 3 1-46.

Borsari, M. et al. J. Chem. Soc. Dalton Trans 1996,4201-4205.

Buchen et al. J. Chem. Soc., Dalton Trans. 1997, 2697-2703.

Budde et al. Neuroscience 1997, 79, 347-358.

Burton et al. J. Soc. Chem. Ind. London 1948: 67: 345

Canzoniero et al. Neurobiology of Disease 1997, 4, 275-279.

Castresana, J. M. et al. Acta Cryst. C 1984, 40, 763-765.

Chenier, P. J. Tetrahedron Lett. 1997, 38, 7341.

Choi et al. Ann. Rev. Neurosci. 1998, 21: 347-375

Copeland et al. J. Am. Chem. Soc. 1999, 121, 4306-4307.

Cuajungco et al. Neurobiology of Disease 1997, 4: 137-169

Czarnik, A. W. Curr. Biol. 1995, 2: 423-428

da Mota et al. J. Chem. Soc. (A) 1969, 2036-2042.

de Silva et al. Chem. Rev. 1997, 97: 1515-1566

Del Zotto, A.; Mezzeffi, A.; Rigo, P. Inorg. Chim. Acta. 1990, 171, 61-69.

Dias, H. V. Inorganic Chem. 1996, 35, 6074.

Doerrer, L. H. Ph.D. dissertation, Massachusetts Institute of Technology, 1997.

Doyle, M. P.; Hoekstra, J. W. J. Inorg. Biochem. 1981, 14, 351-358.

Eaton, D. R. Inorganic Chem. 1968, 7, 2040.

Ebadi, et al. Methods Enzymol. 1991, 205: 363-387

Ebadi, et al. J. Neurochem. 1996, 66: 2121-2127

Ebadi, et al. Neurochem. Int. 1995, 27: 1-22

Evans, I. J. Org. Chem. 1959, 24: 863

Fabbrizzi, L. et al. Angew. Chem. Int. Ed. 1998, 37, 800-802.

Fabbrizzi, L.; Licchelli, M.; Pallavicini, P. Acc. Chem. Res. 1999, 32, 846-853.

Fahrni et al. J. Am. Chem. Soc. 1999, 121, 11448-11458.

Feig et al. Inorg. Chem. 1996, 6892-6898

Flack, H. D. Acta Cryst. 1983, A39, 876-881.

Franz, K. J.; Singh, N.; Lippard, S. J. Angew Chem. Int. Ed. 2000, 39, 2120.

Frederickson et al. Biol. Signals 1994, 3: 127-139

Frederickson et al. J. Neurosci. Meth. 1987, 20, 91-103.

Frederickson, C. Int. Rev. Neurobiol. 1989, 31: 145-238

Furchgott, R. F. Angew. Chem. Int. Ed. 1999, 38, 1870-1880.

Gruenwedel, D. W. Inorg. Chem. 1968, 7: 495-501

Haider, S. Z. et al. Inorg. Chim. Acta 1983, 72, 21-27.

Harrison et al. Neuropharmacology 1994, 33: 935-952

Hartwig et al. Acc. Chem. Res. 1998, 31, 852-860.

Hirano et al. Angew. Chem. Int. Ed. 2000, 39, 1052-1054.

Hörlein, U. Chemische Berichte 1954, 87, 463-472.

Houser et al. J. Am. Chem. Soc. 1995, 117, 10745-10746.

Huang, E. Proc. Natl. Acad. Sci. U.S.A. 1997, 94: 13386-13387

Ignarro, L. J. Angew. Chem. Int. Ed. 1999, 38, 1882-1892.

Jia, L. et al. Nature 1996, 380, 221-226.

Jin, J. Y.; Miwa, T.; Mao, L. Q.; Tu, H. P.; Jin, L. T. Talanta 1999, 48, 1005-1011.

Katayama, Y.; Takahashi, S.; Maeda, M. Anal. Chim. Acta 1998, 365, 159-167.

Koike et al. J. Am. Chem. Soc. 1996, 118, 12696-12703.

Koike, T. et al. J. Am. Chem. Soc. 1992, 114, 7338-7345.

Kojima, H. et al. Anal. Chem. 1998, 70, 2446-2453.

Kojima, H. et al. Angew. Chem. Int. Ed. 1999, 38, 3209-3212.

Kojima, H. et al. Chem. Pharm. Bull. 1998, 46, 373-375.

Kovacs, Z.; Sherry, A. D. Tet. Lett. 1995, 51, 9269-9272.

Lakowicz, J. R. Principles of Fluorescence Spectroscopy 2^{nd} ed.; Kluwer Academic/Plenum Publishers, 1999.

Lancaster, J. R. Nitric Oxide-Biol. Chem. 1997, 1, 18-30.

Lippard et al. Principles of Bioinorganic Chemistry; 1st ed.; University Science Books: Mill Valley, 1994.

Mahadevan et al. Aust. J. Chem. 1996, 49, 561-568.

Malinski, T. et al. Biochem. Biophys. Res. Commun. 1993, 193, 1076-1082.

McBryde, W. A. E. Talanta 1974, 21: 979-1004

McClellan J. Am. Chem. Soc. 1966, 88, 5165.

Meineke, P. et al. Chem. Eur. J. 1999, 5, 1738-1747.

Methods in Enzymology; Packer, L., Eds.; Academic Press: Boston, 1996.

Minta, A.; Kao, J. P. Y.; Tsien, R. Y. J. Biol. Chem. 1989, 264, 8171-8178.

Murad, F. Angew. Chem. Int. Ed. 1999, 38, 1856-1868.

Nasir, et al. JBIC 1999, 4: 775-783

Nozoe, T. J. Chem. Research (S) 1997, 362.

Palmiter et al. EMBO J. 1995, 14: 639-649

Palmiter, et al. EMBO J. 1996, 15: 1784-1791

Palmiter, et al. Proc. Natl. Acad. Sci. USA 1992, 89: 6333-6337

Palmiter, et al. Proc. Natl. Acad. Sci. USA 1996, 93: 14934-14939

Pfeiffer, S.; Mayer, B.; Hemmens, B. Angew. Chem. Int. Ed. 1999, 38, 1714-1731.

Pountney, et al. FEBS Lett. 1994, 345: 193-197

Prasad et al. J. Chem. Soc. Perkin Trans. 1991, 3329-3332.

Rieth, R.; Sasamoto, K. Anal. Commun. 1998, 35, 195-197.

Romary et al. J. Chem. Soc (C) 1968, 2884-2887.

Sato et al. Synthesis 1992, 539-540.

Sen, et al. J. Indian Chem. Soc. 1929, 6, 51.

Shaughnessy et al. J. Am. Chem. Soc. 1999, 121, 2123-2132.

Slomianka, L. Neuroscience 1992: 48, 325-352

Slone, C. S.; Mirkin, C. Prog. Inorg. Chem. 1999, 48, 233.

Smith, et al. J. Chem. Soc. Perkin Trans. 1993, 1195-1204.

Steinheubel, D. P. Inorganic Chem. 1999, 38, 6225.

Steinheubel, D. P. Organometallics 1999, 18, 3959.

Sun et al. J. Org. Chem. 1997, 62, 6469-6475.

Trevin, S.; Bedioui, F.; Devynck, J. Talanta 1996, 43, 303-311.

Tsien, R. Y. Ann. Rev. Neurosci. 1989, 12, 227-253.

Tsien, R. Y. Biochemistry 1980, 19, 2396-2404.

Tsien, R. Y. Fluorescent and Photochemical Probes of Dynamic Biochemical Signals Inside Living Cells; Czarnik, A. W., Ed.; American Chemical Society: Washington D.C., 1993; Vol. 538, pp 130-146.

Tsuji, et al. EMBO J. 1992, 11: 4843-4850

Uchida, et al. Neuron 1991, 7: 337-347

Uhlig, V. E. et al. Z. Anorg. Allg. Chem. 1982, 492, 52-62.

Vallee et al. Physiol. Rev. 1993, 73: 79-118

Walkup et al. J. Am. Chem Soc. 2000, 122: 5644-5645

Walkup et al. J. Am. Chem Soc. 2000: 122: S1-S7

Wolf, H. U. Experientia 1973, 29: 241-249

Wolfe et al. J. Org. Chem. 2000, 65, 1144-1157.

Wolfe et al. J. Org. Chem. 2000, 65, 1158-1174.

Wood, J.; Garthwaite, J. Neuropharmacology 1994, 33, 1235-1244.

Zalewski et al. Biochem. J. 1993, 296, 403-408.

Zask, A. Inorganic Chem. 1986, 25, 3400.

Zhang et al. J. Org. Chem. 2000, 65, 8027-8031.

U.S. Pat. Nos. 6,051,207, 6,013,802, 6,083,758, 6,063,637, 5,986,094, 5,756,771, 5,721,365, 5,346,670 and 4,510,251; U.S. Ser. No. 09/901,466 (filed Jul. 9, 2001); and U.S. Ser. No. 10/124,742 (filed Apr. 17, 2002).

Equivalents

The invention now being fully described, it will be apparent to one of ordinary skill in the art that many changes and modifications may be made thereto without requiring more than routine experimentation or departing from the spirit or scope of the appended claims.

We claim:

1. A coordination complex for detecting an analyte, comprising:
(a) a first metal ion in a coordination geometry having three or more equatorial coordination sites and one or more axial coordination sites,
(b) three or more Lewis base atoms coordinated to the first metal ion, wherein the Lewis base atoms are in equatorial coordination sites of the first metal ion, and
(c) a ligand V—F, wherein V is a metal binding domain coordinated to the first metal ion in an axial coordination site of the metal ion and F is a fluorophore, wherein the fluorescence intensity of a sample of the coordination complex increases upon exposure to an analyte.

2. The coordination complex of claim 1, wherein the analyte is NO.

3. The coordination complex of claim 1, wherein the coordination complex comprises at least one metal ion in addition to the first metal ion.

4. The coordination complex of claim 1, wherein the coordination geometry of the first metal ion is octahedral.

5. The coordination complex of claim 1, wherein at least three Lewis base atoms coordinated to the first metal ion in equatorial coordination sites are from the same macrocycle.

6. The coordination complex of claim 5, wherein the three Lewis base atoms are nitrogen.

7. The coordination complex of claim 1, wherein at least three of the Lewis base atoms coordinated to the first metal ion in equatorial coordination sites are each derived from a different bidentate anionic ligand.

8. The coordination complex of claim 7, wherein each of the bidentate anionic ligands is a carboxylate.

9. The coordination complex of claim 7, wherein each of the bidentate anionic ligands is the same carboxylate.

10. The coordination complex of claim 7, wherein the first metal ion is a transition metal ion.

11. The coordination complex of claim 1, wherein the fluorophore F comprises rhodafluor.

12. The coordination complex of claim 1, wherein F is a derivative of dansyl.

13. The coordination complex of claim 1, wherein the ligand V—F is covalently tethered to one of the ligands that contributes at least one of the three Lewis base atoms coordinated to the first metal ion in an equatorial coordination site.

14. The coordination complex of claim 1, wherein the coordination complex in the sample is in solution.

15. The coordination complex of claim 1, wherein in addition to an increase of the fluorescence intensity of a sample of the coordination complex upon exposure to an analyte, there is a change in one or more of the following upon such exposure: the emission wavelength and the excitation wavelength.

16. The coordination complex of claim 1, wherein the increase of the fluorescence intensity of a sample of the coordination complex upon exposure to an analyte arises substantially from the metal binding domain V of the ligand V—F no longer being coordinated to a metal ion of the coordination complex to the same extent after the exposure as compared to before the exposure.

17. A coordination complex for detecting an analyte, comprising the moiety {M(MC)(V—F)}, wherein: MC represents a macrocycle that is capable of coordinating a metal ion through at least two Lewis basic atoms; M is a metal ion; V is a metal binding domain that is capable of forming a coordinate bond with M; and F is a fluorophore; and wherein the fluorescence intensity of a sample of the coordination complex increases upon exposure to an analyte.

18. The coordination complex of claim 17, wherein the moiety {M(MC)(V—F)} is charged.

19. The coordination complex of claim 17, wherein the MC coordinates the metal ion through four Lewis base atoms that are contained in a single ring moiety.

20. The coordination complex of claim 17, wherein the MC is porphyrin or a porphyrin-based ligand.

21. A coordination complex for detecting an analyte, comprising: {Mₘ(W)ₙ(V—F)ₚ}, wherein independently for each occurrence: W represents a ligand which is capable of coordinating one or more metal ions through at least two Lewis basic atoms; M is a metal ion; V is a metal binding domain that is capable of forming a coordinate bond with M; F is a fluorophore; m is at least 2; n is independently 3 or 4; and p is independently 1 or 2.

22. The coordination complex of claim 21, wherein the moiety {Mₘ(W)ₙ(V—F)ₚ} is charged.

23. The coordination complex of claim 21, wherein all W are carboxylate ligands.

24. The coordination complex of claim 21, wherein m is 2, n is 4, all W are the same carboxylate ligand, and p is 2.

25. The coordination complex of claim 21, wherein m is 2 and each M is the same transition metal ion.

26. The coordination complex of claim 21, wherein the fluorescence intensity of a sample of the coordination complex increases upon exposure to an analyte.

27. A method of detecting, and optionally quantifying the concentration of, an analyte in a sample, comprising:
a. Adding to a sample one or more the coordination complexes of any of the preceding claims;
b. Measuring the fluorescence of the sample; and
c. Determining whether the analyte is present in the sample, and optionally the concentration of the analyte in the sample.

28. A kit for detecting an analyte, comprising one or more the coordination complexes of any of the preceding claims and instructions for using the coordination complex to detect an analyte.
